(19) United States
(12) Patent Application Publication
(10) Pub. No.: US 2010/0016731 A1
(43) Pub. Date: Jan. 21, 2010

(54) Hemodynamic Detection of Circulatory Anomalies

(75) Inventors: Philip E. Eggers, Dublin, OH (US); Andrew R. Eggers, Ostrander, OH (US); Eric A. Eggers, Portland, OR (US)

Correspondence Address: MUELLER AND SMITH, LPA;MUELLER-SMITH BUILDING, 7700 RIVERS EDGE DRIVE, COLUMBUS, OH, 43235, US

(73) Assignee: Cardiox Corporation, Dublin, OH (US)

(21) Appl. No.: 12/418,866

(22) Filed: Apr. 6, 2009

Related U.S. Application Data
(60) Provisional application No. 61/156,723, filed on Mar. 2, 2009.
(60) Provisional application No. 61/080,724, filed on Jul. 15, 2008.

(51) Int. Cl. A61B 5/05; A61B 5/097; A61B 5/02; A61B 6/00

(52) U.S. Cl. 600/476; 600/484; 600/504; 600/407; 600/508

(57) ABSTRACT

The preferred indicator is an injection of indocyanine green dye which is detected and quantified by causing it to fluoresce at a sensor location, for example, at the human ear. Quantification is carried out by using cardiac output procedures and where called for, the Valsalva Maneuver is monitored at a monitor/controller giving visual cues to the patient and operator as to the quality of the necessary exhalation pressure maintenance.

### CROSS-REFERENCE TO RELATED APPLICATIONS

This application claims the benefit of provisional Application No., 61/156,723, filed Mar. 2, 2009, and provisional Application No., 61/080,724 filed Jul. 15, 2008, the disclosures of which are incorporated by reference.

### STATEMENT REGARDING FEDERALLY SPONSORED RESEARCH

None

### BACKGROUND OF THE INVENTION

The present invention generally relates to a system, method and apparatus for detection of circulatory anomalies in the mammalian body. Important ones of such anomalies are generally referred to as cardiac right-to-left shunts.

An anomaly commonly encountered in humans is an opening between chambers of the heart, particularly an opening between the left and right atria, i.e. a right-left atrial shunt, or between the left and right ventricles, i.e. a right-left ventricular shunt. The shunt may occur as a defect within the vasculature leading to and from the heart, for example a Pulmonary Arteriovenous Malformation (PAVM) may be present as an open hole shunting between vein and artery. Over 780,000 patients suffer strokes each year in the U.S. resulting in 250,000 stroke related deaths. The total cost associated with stroke is reported to be $66 billion in the U.S. in 2007 (Rosamond 2008). Of the patient population presenting with stroke or the early warning sign known as transient ischemic attack (TIA or mini stroke), as many as 260,000 are reported to be the result of a right-to-left shunt in the heart and/or pulmonary vasculature.

The most common form of right-to-left shunt is the patent foramen ovale (PFO) which is an opening in the wall of the heart which separates the right side of the heart from the left side of the heart. The right side of the heart receives oxygen-depleted blood from the body and then pumps this blood into the lungs for reoxygenation. The lungs not only reoxygenate the blood but also serve as a “filter” for any blood clots and also serves to metabolize other agents that naturally reside within the venous blood. During the fetal stage of development, an opening naturally exists between the right and left side of the heart to enable circulation of the mother's oxygen-rich blood throughout the vasculature of the fetus. This opening between the right and left side of the fetus' heart (known as the foramen ovale) permanently seals shut in consequence of the closure of a tissue flap in about 80% of the population within the first year following birth. Often the noted flap remains in a sealing orientation because of a higher pressure at the left side of the heart. However, in the remaining 20% of the population, this opening fails to permanently close which is referred to as a patent foramen ovale or PFO.

Most of the population exhibiting a PFO never experience any symptoms or complications associated with the presence of a PFO since many PFOs are small enough to remain effectively “closed.” However, for some subjects, this normally closed flap (i.e., foramen ovale) temporarily opens allowing blood to flow directly from the right side to the left side of the heart. As a consequence, any blood clots or other active agents escaping through the PFO bypass the critical filtering functions of the lungs and flow through the brief opening in this flap and directly to the left side of the heart. Once in the left side of the heart, any unfiltered blood clots or metabolically active agents pass directly into the arterial circulatory system. Since a significant portion of the blood exiting the left side of the heart flows to the brain, any unfiltered blood clots or agents such as serotonin may be delivered to the brain. Presence of these substances in the brain arterial flow can produce debilitating and life-threatening consequences. These consequences are known to include stroke, heart attack and are also now believed to be one of the causes of certain forms of severe migraine headaches. For further background on circulatory anomalies, see:
1) Banas, J., et al. American Journal of Cardiology 28: 467-471(October 1971);
2) Castillo, C., et al. American Journal of Cardiology 17: 691-694 (May 1966);
3) (Schwedt 2006, Weinberger 2007);
4) (Spies 2006, Wammes 2006).

A relatively large number of patients (three million) have or may be undergoing sclerotherapy treating, for instance, varicose veins. This therapy involves an injection of sclerosing solution which in effect creates emboli. If patients undergoing sclerotherapy are among the proportion of the population with a PFO, creation of emboli that may bypass the filtering aspect of the lungs creates a significant risk of initiating a TIA, stroke or heart attack. This risk could be avoided by effectively and efficiently screening for a right-to-left shunt.

Based on the growing clinical evidence linking strokes, transient ischemic attacks (TIAs) and migraine headaches to right-to-left shunts, at least 16 companies have now entered the field of transvascular shunt treatment devices for closure of the most common form, viz., a patent foramen ovale (PFO), and certain of these devices are approved for sale in one or more principalities.

Percutaneous closure devices are expected to soon be widely available in the U.S. for PFO closure, and over 10% of the adult population is estimated to have a congenital patent foramen ovale (PFO). Unfortunately, there is currently no available method suitable for widespread screening for the presence of a PFO when the patient experiences early warning signs signaling an ischemic incident, or the patient exhibits or is exposed to an elevated risk of a stroke. Consequently, the “at risk” fraction of the population with a right-to-left shunt is most often resigned to the possibility of experiencing a stroke before definitive right-to-left shunt testing is performed. Only then are methods such as transesophageal echocardiography (TEE) performed to detect the possible presence of a right-to-left shunt. If detected, the patient may elect one of a growing number of transcatheter right-to-left shunt closure procedures or the more conventional open-heart procedure for right-to-left shunt closure.

Transesophageal echocardiography (TEE) is resorted to somewhat as a last resort. It is considered the “gold standard” of determining the presence of a right-to-left shunt. In carrying out this test, microbubbles are injected into a vein leading to the right side of the heart. As this is underway, the patient is required to blow into a manometer to at least a pressure of 40 mm of mercury (Valsalva maneuver). Simultaneously, a sonic detector is held down the throat to record the passage of the microbubbles across the shunt. Because of gagging problems, the patient is partially anesthetized. Typically, patients will refuse to repeat the painful test and it is hardly suited for screening. The TEE test is expensive with an equipment total cost of between $75,000 and $322,000. It additionally requires a physician with a specialized two year fellowship and an anesthesiologist.

Another test is referred to as transthoracic echocardiography (TTE). Again, microbubbles are injected into a vein leading to the right side of the heart. The Valsalva maneuver is carried out and ultrasonic echograms are made at the chest wall. The procedure requires the use of expensive equipment and exhibits about a 60% sensitivity.

A third test again uses microbubbles as a contrast agent along with the Valsalva maneuver. Here, however, the ultrasonic sensors perform in conjunction with the temporal artery usually at both sides of the head. This transcranial doppler method (TCD) exhibits a high sensitivity and costs between about $30,000 to $40,000 for equipment. Unfortunately, over 20% of the population has a cranial bone that's too thick for sonic transducing. U.S. Patent Publication US2006/0264759 describes such systems and methods for grading microemboli in blood associated with ultrasound contrast agenda (e.g., small air bubbles) within targeted vessels by using Doppler Ultrasound system.

Additional description of existing methods of analyzing circulation and detecting certain circulatory anomalies are present in the following.
5) Swan, H., et al. Circulation X: 705-713(November 1954);
6) Kaufman, L., et al. Investigative Radiology 7: 365-368(September-October 1972);
7) Karttunen, V., et al. Acta Neurologica Scandinavica 97: 231-236 (1998);
8) Karttunen, V., et al. Stroke 32: 445-453 (2001).

A continuing difficulty with existing methods is the efficacy of using microbubbles as a circulatory tracking indicator. Microbubbles are created just prior to use, are a transient structure, and decidedly non-uniform in creation and application. It is difficult if not impossible for microbubbles to be used for quantitative measurements, and thus clinicians are forced to rely on a positive or negative result assessment. In part, the inability to effectively quantify the conductance of a shunt is revealed in the relatively low sensitivity of the existing methods.

A further problem with existing methods is the difficulty in effectively detecting the circulatory tracking indicator in the form of microbubbles. Each of existing methods, including transesophageal echocardiography, transthoracic echocardiography, and the transcranial doppler method suffer from barriers for routine use for screening, whether due to the need for anesthesia or expensive equipment. There is a need for more efficient circulatory tracking reagents, i.e. a reagent that can be reproducibly introduced into the circulatory system, be quantitatively detectable, and utilize relatively straightforward detection systems that are easily tolerated by patients.

One difficulty with improving the present technology in circulatory tracking reagents is that there heretofore has been no animal model available for screening a variety of different circulatory tracking reagents and their compatible detection systems.

There exists a growing body of clinical evidence linking the presence of right-to-left shunts to the risk of embolic strokes and occurrence of migraine headaches. In spite of this evidence, there remains a significant unmet need for a high sensitivity, low-cost and non-invasive method to screen those patients at increased risk of stroke in order to detect PFOs or other circulatory anomalies. The ability to screen at-risk patients is a critically unmet need, since shunt-related strokes can only be prevented if the presence of the shunt is detected and closed in advance of the occurrence of a stroke. In addition, there is likewise a significant unmet need for a highly sensitive, quantitative low-cost method for evaluating the effectiveness and durability of the closure at 3 to 4 time points following the percutaneous closure of the right-to-left shunt. This follow-up testing following shunt closure continues to be essential for assuring adequacy of the “seal” closing a PFO or other shunt, in order to minimize the risk of future shunt-related strokes.

Thus there is an unmet need for a reliable, quantitative system for detecting and evaluating circulatory anomalies. In addition there is a need for a system for evaluating circulatory tracking reagents and compatible detection apparatus that could be utilized for assessing circulatory anomalies and other circulatory phenomena.

### BRIEF SUMMARY

The present system is addressed to system, method and apparatus for detecting and quantifying right-to-left cardiac shunts. The preferred indicator which is employed is indocyanine green dye (ICG) which will fluoresce when exposed to an appropriate wavelength of higher energy light, for example, a laser in the red region. The procedure is under the control of a monitor/controller having a visual display and capable of providing cues to both the operator and the patient. A vein access catheter is employed in connection with a peripheral vein such as the antecubital vein in an arm. This delivery system utilizes a unique resistance feedback-controlled heater-type fluid sensor to control injection times for both the indicator and a predetermined volume of isotonic saline used to “flush” flow sensor extension tubing, the venous catheter and peripheral vein so that all injected indicator is promptly delivered to the right atrium of the heart.

Sensing of the indicator concentration takes place at an arterial vasculature, for example, the pinna of the human ear. Additionally, heart rate is monitored.

Where a test is to be carried out with a Valsalva Maneuver, the mouthpiece of a manometer tubing set is positioned in the mouth of the patient and connected to a pressure transducer in the monitor/controller.

A visual readout of instantaneous exhalation pressure is made available both to the operator and the patient as well as the threshold level of pressure which must be achieved and maintained in order to successfully carry out a Valsalva Maneuver. Alarms and visual error messages are introduced where this Valsalva Maneuver is not carried out properly. The monitor/controller instructs the operator with an audible cue to inject indicator and immediately inject isotonic saline flush. These are generally carried out with two syringes working in conjunction with a three-way valve. If the injection interval is not appropriate, again an audible alarm and error message is provided to the operator. For the relief of the patient, an audible cue also is given when the Valsalva Maneuver can be stopped.

The monitor/controller then calculates average heart rate and cardiac output, using that average heart rate, calculated body surface area, and a known normal value for stroke index of the heart. The system then calculates an area under a normal indicator/dilution curve associated with indicator and blood flow through a normal pathway in the lungs. Additionally, the monitor/controller calculates the area under any premature indicator dilution curve which will be associated with a right-to-left shunt. The monitor/controller further corrects the main indicator/dilution curve for a recirculation phenomenon and to quantify any right-to-left shunt calculated conductance associated with such shunts.

Other objects of the invention will, in part, be obvious and will, in part, appear hereinafter. The invention, accordingly, comprises the method, apparatus and system possessing the construction, combination of elements, arrangement of parts and steps which are exemplified in the following detailed description.

For a full understanding of the nature and objects of the invention, reference should be made to the following detailed description taken in connection with the accompanying drawings.

### BRIEF DESCRIPTION OF THE DRAWINGS

FIG. 1 is a schematic representation of a heart showing a right-to-left heart shunt;

FIG. 1A is an enlargement of a portion of the schematically illustrated heart of FIG. 1.

FIG. 2 is a chart showing excitation and fluorescence signal attenuation for depth of artery versus wavelength in connection with two regulatory approved dyes;

FIG. 3 is a schematic end view of a fluorescence probe used for bench-top and animal testing;

FIG. 4 is a schematic representation of the end geometry utilized with the probe shown in FIG. 3;

FIG. 5 is a schematic representation of bench-top test apparatus employed for optimization of a fluorescence detection system;

FIG. 6 is a representation of the display of a digital oscilloscope used in bench-top testing;

FIG. 7 is a representation of measured fluorescence peak signal amplitudes with respect to the excitation level of a laser;

FIG. 8 is a pictorial representation of a test being undertaken under the protocol of the invention;

FIG. 9 is a schematic representation of a right-to-left shunt test according to the invention;

FIG. 10 is a representation of a display output showing concentration of indicator in blood, measured exhalation pressure level and interval of injection of indicator;

FIG. 11 is a schematic representation of the invention showing two shunt conditions;

FIG. 12 is a display relating concentration of indicator in blood, measured exhalation pressure level and the elapsed time after indicator injection corresponding with the schematic representation of FIG. 11;

FIG. 13 is a graph relating concentration of indicator with time;

FIG. 14 is a graph relating indicator signal amplitude with respect to elapsed time since the start of an injection and further showing a recirculation effect;

FIG. 15 is a view of a display seen in FIG. 8;

FIG. 16 is an assembly view of the indicator injection system of the invention;

FIG. 17 is an exploded view of a fluid sensor employed with the system of FIG. 16;

FIG. 18 is a side view of the fluid sensor employed with the invention;

FIG. 19 is a sectional view taken through the plane 19-19 in FIG. 18;

FIG. 20 is a front view of a printed circuit employed with the fluid sensor of the invention;

FIG. 21 is a perspective view of a pipe employed with the fluid sensor of the invention;

FIG. 22 is a perspective view of joined enclosure halves of the fluid sensor of the invention;

FIG. 23 is a front view of a connector employed with the fluid sensor of the invention;

FIG. 24 is a sectional view taken through the plane 24-24 in FIG. 23;

FIG. 25 is a front view of a connector employed with the fluid sensor of the invention;

FIG. 26 is a sectional view taken through the plane 26-26 in FIG. 25;

FIG. 27 is an assembly view of a sensor employed with the invention;

FIG. 28 is an anatomical view of a human ear showing arterial vasculature;

FIG. 29 is a top view of the sensor shown in FIG. 27;

FIG. 30 is a sectional view taken through the plane 30-30 shown in FIG. 29;

FIG. 31 is an assembly view of a system for carrying out the Valsalva Maneuver;

FIG. 32 is a block schematic diagram showing the components of a monitor/controller/data recorder according to the invention;

FIG. 33 is a block schematic view of an animal test arrangement;

FIG. 34 is a schematic view of a heart of a pig animal as employed with the animal test of the invention;

FIG. 35 is a graph showing signal amplitude versus elapsed time developed with the animal studies of the invention;

FIG. 36 is a plot of peak signal and concentration area for an initial twenty tests described in Table 1;

FIG. 37 is a plot similar to that at FIG. 36 but showing a second twenty tests undertaken as listed in Table 1; and

FIGS. 38A-38F combine as labeled thereon to illustrate the methods and procedures of the invention.

### DETAILED DESCRIPTION OF THE INVENTION

The case of a flow system with two or more alternative flow pathways exists in the human body when a right-to-left shunt is present in the heart or the pulmonary circulation. As described above, most common form of a right-to-left shunt in the heart is known as a Patent Foramen Ovale or PFO. During the fetal stage of development, an opening naturally exists between the right and left side of the heart to enable circulation of the mother's oxygen-rich blood throughout the vasculature of the fetus. This opening between the right and left side of the fetus' heart (known as the foramen ovale) permanently seals shut in about 80% of the population within the first year following birth. However, in the remaining 20% of the population, this opening fails to permanently close which is referred to as a Patent Foramen Ovale or PFO.

For some individuals, this normally closed flap (i.e., Foramen Ovale) temporarily opens allowing blood to flow directly from the right side to the left side of the heart. As a consequence, any blood clots or other metabolically active agents such as serotonin bypass the critical filtering/metabolic functions of the lungs and flow through the brief opening in this flap and directly to the left side of the heart. Once in the left side of the heart, any unfiltered blood clots or agents such as serotonin pass directly into the circulatory system. Since a portion of the blood exiting the left side of the heart flows to the brain as well as the coronary arteries of the heart, any unfiltered blood clots or agents such as serotonin can produce debilitating and life-threatening consequences. These consequences are known to include stroke, heart attack and are also now believed to be one of the principal causes of certain forms of severe migraine headaches.

For further discussion, see:
9) Spies C., et al., “Patent Foramen Ovale Closure With the Intrasept Occluder: Complete 6-56 Months Follow-Up of 247 Patients After Presumed Paradoxical Embolism,” Catheterization and Cardiovascular Interventions 71: 390-395 (2008);
10) Wammes-van der Heijden E. A., et al., “Right-to-left shunt and migraine: the strength of the relationship,” Cephalalgia; 26: 208-213 (2006);
11) Schwedt T. J., et al., “Patent Foramen Ovale and Migraine—Bringing Closure to the Subject,” Headache 2006 46: 663-671 (2006)
12) Weinberger J., “Stroke and Migraine,” Current Cardiology Reports 2007; 9: 13-(2007).

A method, apparatus, and system are described herein for effectively monitoring subject patients for circulatory anomalies. The present method, apparatus, and system are useful for determining the magnitude of the flow rate associated with a right-to-left shunt in the heart and/or within the pulmonary vasculature, for instance. In a preferred embodiment, an optical sensor is positioned on the surface of the subject's skin at a location (e.g., the auricle of the ear). A biocompatible indicator is next injected at a predetermined rate into a peripheral vein of the subject while the subject may be directed to engage in a breathing maneuver, exhaling into a manometer mouthpiece. A pressure differential may be effective for causing the opening of a PFO, for instance, allowing blood to flow across a right-to-left cardiac shunt. A non-invasive optical sensor is used to transcutaneously measure the concentration of the injected indicator as a function of time. As seen in below in connection with FIG. 10, if a premature inflection or peak occurs in the indicator concentration level at a time point, t₁ prior to the rise and fall of the concentration associated with the majority of the indicator flowing through the normal pathway of the lungs and arriving at the sensor at time point t₂, then a right-to-left shunt (e.g., PFO) is present in the heart. As described herein the system apparatus and method provide for calculating and quantifying the extent of circulatory anomalies in a manner superior to any available, and capable of providing diagnostics effective for determining the appropriate clinical treatment of circulatory anomalies.

In the discourse to follow research activities are somewhat tracked as the invention developed. In this regard, bench tests are described looking to basic studies that lead to subsequent animal (pig) tests. Fundamentals of measuring cardiac output lead to the evolution of a method and system wherein right-to-left shunts could not only be detected but also quantified. Effective utilization of the system requires an indicator, i.e. a circulatory tracking reagent, or an analyte capable of passing through the lungs, and which provide information with respect to the left atrium of the heart. The previous systems for detecting PFOs relying on ultrasonic detection of microbubbles, are almost completely incapable of providing information on the size of as PFO, and require semi-invasive detection methods, such as trans-esophageal ultrasound. The inventors had earlier studied techniques for monitoring total circulating blood volume and cardiac output as described at U.S. Pat. No. 6,299,583 by Eggers, et al., issued Oct. 9, 2001, and incorporated herein by reference.

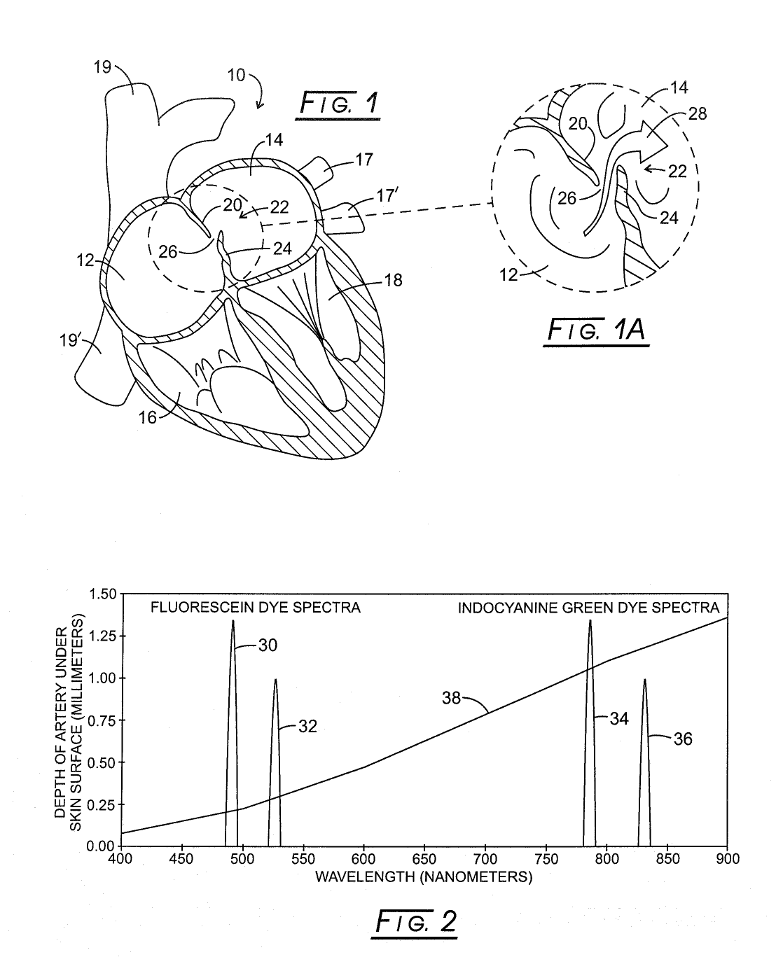

Referring initially to FIG. 1, a mammalian heart is schematically represented and identified in general at 10. The right atrium is shown at 12 and correspondingly, the left atrium is represented at 14. Beneath the right atrium 12 is the right ventricle 16 which is located adjacent to the left ventricle 18. An interauricular septum 20 separates atria 12 and 14 and is shown in FIG. 1A in enlarged fashion to illustrate a PFO represented generally at 22. Typically, venous blood enters the heart through the superior vena cava and inferior vena cava, 19 and 19′, feeding the right atrium, to the right ventricle and pulmonary artery passing to the lungs. From the lungs, the left atrium is supplied with oxygenated blood via the pulmonary veins 17 and 17′, with that blood then being pumped throughout the arterial system by the left ventricle 18 to the aorta, (not shown in FIG. 1). As illustrated in FIG. 1, the atypical presence of a patent foramen ovale 22 results from, for example, the presence of displaceable tissue flap 24, creating opening 26. Shunt flow of venous blood from the right to left atrium through opening 26 is represented by the arrow 28. Shunt flow thus does not pass through the lungs, bypassing the pulmonary circulatory circuit, and potentially allowing detrimental blood components to bypass the filtering capabilities of the lung capillary beds.

The present disclosure provides for a system and method of detecting and quantifying atypical or abnormal blood flow, particularly left to right atrial shunts, and other arterial-venous malformations and disruptions in typical blood circulation. The method relies on effectively quantifying the relative blood flow or blood flow volume passing through normal and aberrant pathways. One example of such aberrant pathways are openings, or shunts between the right and left atria. In order to provide for effective quantification of shunts between arterial and venous blood flow, an indicator, i.e. a circulatory tracking reagent, is needed. Essentially at the outset of the study, it was determined that a high degree of sensitivity would be achieved in screening capabilities for detecting atypical blood flow through the utilization of a fluorescing moiety as an indicator, i.e. a circulatory tracking reagent, in a cardiac output (CO) emulating system. Accordingly, fluorescing dyes were examined and, in particular, fluorescing dyes which have been approved for use in humans. Two such exemplary dyes were available, fluorescein and indocyanine green dye (ICG).

As discussed further below, a number of additional circulatory tracking reagents are available for use with the system, including such indicators as spectrophotometric, densitometric and radiometric indicators. A variety of previous efforts to utilize circulatory tracking indicators include the following. U.S. Pat. No. 3,412,728 describes a method and apparatus for monitoring blood pressure, utilizing an ear oximeter clamped to the ear to measure blood oxygen saturation using photo cells which respond to red and infrared light. U.S. Pat. No. 3,628,525 describes an apparatus for transmitting light through body tissue for purposes of measuring blood oxygen level. U.S. Pat. No. 4,006,015 describes a method and apparatus for measuring oxygen saturation by transmission of light through tissue of the ear or forehead. U.S. Pat. No. 4,417,588 describes a method and apparatus for measuring cardiac output using injection of indicator at a known volume and temperature and monitoring temperature of blood downstream. This and similar art suffer from an inability to effectively quantify the magnitude, i.e., functional conductance, of shunts in part because of the failure and or inability to effectively quantify cardiac output.

A number of patents describe potential reagent systems that if adapted, could be utilized with the present systems method and apparatus. U.S. Pat. No. 4,805,623 describes a spectrophotometric method used for quantitatively determining concentration of a dilute component in an environment (e.g., blood) containing the dilute component where the dilute component selected from group including corporeal tissue, tissue components, enzymes, metabolites, substrates, waste products, poisons, glucose, hemoglobin, oxy-hemoglobin, cytochrome. The corporeal environment described includes the head, fingers, hands, toes, feet and earlobes. Electromagnetic radiation is utilized including infrared radiation having a wavelength in the range of 700 to 1,400 nanometers. U.S. Pat. No. 6,526,309 describes an optical method and system for transcranial in vivo examination of brain tissue (e.g., for purpose of detecting bleeding in the brain and changes in intracranial pressure), including the use of a contrast agent to create image data of the examined brain tissue.

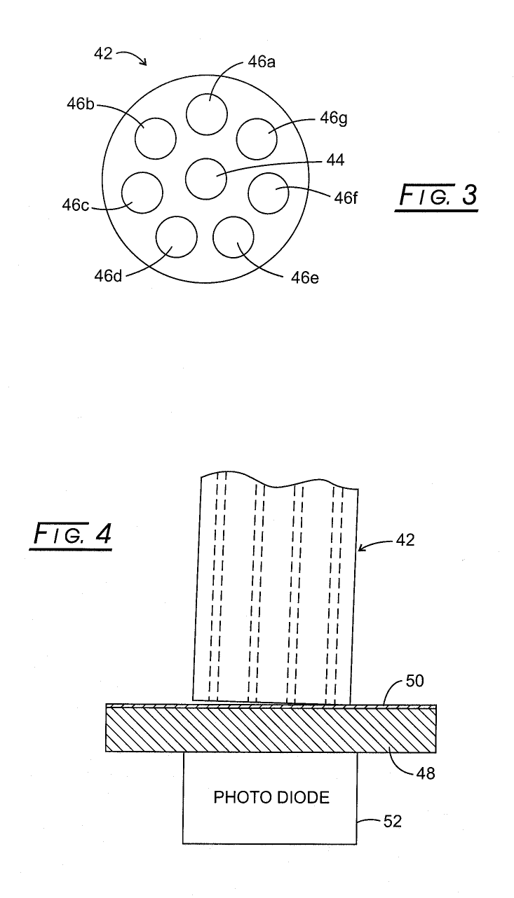

Detailed examination of the desirable properties of fluorescein and indocyanine green demonstrate how other indicators can be applied to the present system and method. These fluorescing dyes are excited by one wavelength of electromagnetic radiation, and emit a detectable signal at a second wavelength. Looking to FIG. 2, the excitation signal for fluorescein dye is represented at curve 30 which is an excitation signal at less than about 500 nanometers (nm). A resultant fluorescence emission from the excitation at 30 is shown at curve 32, at a wavelength of about 525 nm.

Looking to the indocyanine green dye spectrum, an excitation curve is shown at 34, having a peak excitation wavelength at about 785 nanometers. Correspondingly, the fluorescence emission from excitation at curve 34 is represented at curve 36 with a peak emission wavelength of about 830 nanometers. Directed across FIG. 2 is a curve representing the level of constant signal attenuation or absorption of about 37% resulting from passage of signal through tissue. That curve is represented at 38. Note that if a blood vessel is about one millimeter beneath the surface of skin the system would perform at about 37% of the original intensity of the excitation signal and fluorescence signal for the case of indocyanine green dye indicator. In contrast, a 37% decrease in excitation and emission signal level would result for the fluorescein dye indicator at a tissue thickness of only 0.25 mm. Since blood vessel being targeted is at least 1 mm below skin surface, the attenuation and associated signal loss would be much larger. Therefore, indocyanine green dye is preferred.

Turning again to the indocyanine green dye spectrum, note that the excitation signal 34 for ICG intersects curve 38 at a depth allowing for readily reaching blood vessels, for example within the pinna of the ear. Accordingly, indocyanine green is compatible with use as a circulatory tracking agent when detecting through skin, and indocyanine green was the indicator elected for subsequent study.

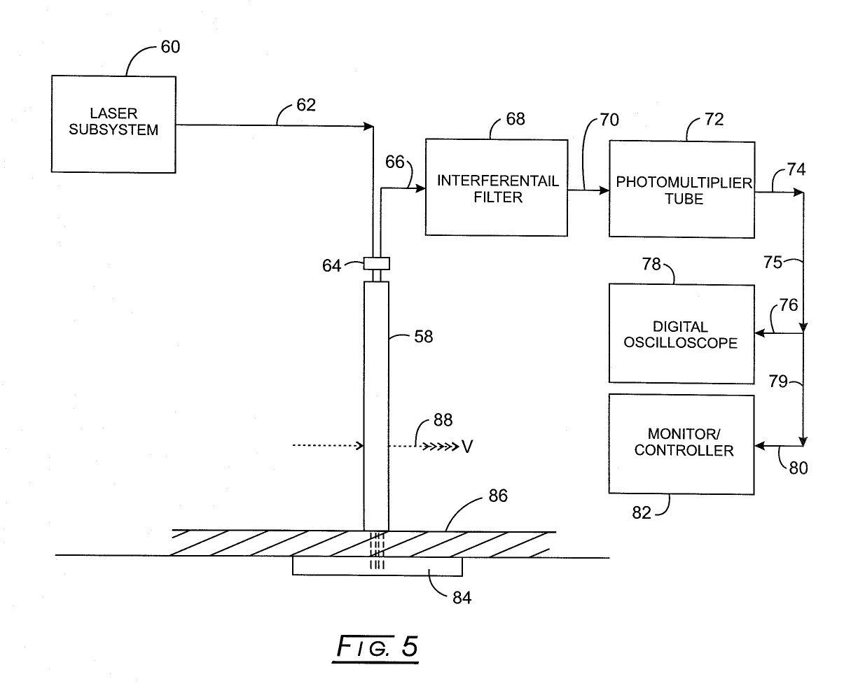

To utilize this fluorescing form of indicator in carrying out cardiac output related procedures, a sensor or probe apparatus having the capability to direct laser excitation illumination to a blood vessel as well as to collect and filter an emitted fluorescent response, a sensor comprising excitation and detection components was developed utilizing fiber optic technology. Looking to FIG. 3, one end of such a sensor, 42, is revealed. At the center of sensor 42 is a fiber optic channel identified at 44 which projects excitation emissions, for example, at 785 nanometers for ICG. Surrounding central fiber 44 are 7 glass fibers identified at 46a-46g. All of these glass fibers have an outside diameter of 600 microns. When an ICG indicator within the bloodstream reaches the site of irradiation with 785 nm (laser) light, the fluorescent moiety within the ICG indicator is excited as described in connection with FIG. 2 to an elevated energy state for a brief period. As the excited moiety returns to its normal energy state, it emits light at a longer wavelength (viz., 830 nm) and the difference between the excitation wavelength (viz., 785 nm) and the fluorescence emission wavelength (viz., 830 nm) is known as the Stokes Shift. This Stokes Shift of nominally 45 nm allows the fluoresce emission to be extracted by using a interferential or other type of optical filter to reflect or otherwise attenuate all but the wavelength band of interest (viz., 820 to 840 nm). It is important that the interface between this interferential filter and the fiber optic sensor be accurately aligned. With alignment, at a preferred angle of about 7 degrees, the filter will reflect unwanted wavelengths. Angles of photon incidence of greater than about 45 must be avoided since interferential filters will become ineffective. Looking to FIG. 4, sensor 42 reappears along with filter 48. Filters, as represented at 48 typically are coated at their entrance with a metal as represented at 50. Metal 50 represents an optical coupling agent known to those skilled in the art of optics and multi-coated optical lenses. It was found beneficial to assure that the alignment was about 7 degrees to avoid undue reflections at angles which would pass the filter. Beyond the filter 48 is a photo detector 52. In general, establishing a quality sensor/filter interface permitted the use of higher laser power levels.

In the course of carrying out the instant studies, a variety of bench top jigs or the like were developed. One such arrangement is revealed in FIG. 5. In the figure, a laser excitation and fluorescence collecting sensor is represented schematically at 58. In this regard, a glass fiber laser input to sensor 58 shown having a source shown at block 60 coupled with a fiber transmitter 62 extending to a spliced connection 64. In turn, connection 64 is coupled to the input of sensor 58. Block 60 incorporates a multi mode laser subsystem marketed as a model No. 785-2P-OEM by Ocean Optics, Inc. Splice connection 64 is a model No. SMA-2FL-ADP. A collected fluorescence output is directed from splice 64 via glass fiber 66 to an interferential filter passing generally 830 nanometers and represented at block 68. Filter 68 was provided as a model No. 679-3230 marketed by OptoSigma, Inc. as represented at line 70 and block 72. Filter 68 is connected such that it is operatively cooperated with a photomultiplier tube 72. Tube 72 was provided as a model No. H7712-10 marketed by Hamanatsu, Inc. Signals from photomultiplier tube 72 during initial test were directed as represented at arrows 74-76 to a phase one digital oscilloscope provided as a model No. DTO3054 marketed by Techtronics, Inc. At a later time, such signals would be further directed as represented at arrows 79 and 80 to a phase 2 monitor/controller 82, comprising a controller/data acquisition system discussed later herein. The transmitting and receiving end of sensor 58 performed in conjunction with a sealed glass capillary cylinder sample tube, e.g., 84, carrying ICG fluorescing dye of given concentrations. In general, such tubes as at 84 have an inside diameter of 1-2 milliliters. To simulate performance transcutaneously, the tube 84 is positioned just below the surface of a human skin phantom material represented at 86. For many of the tests, sensor 58 was moved along tube 84 or transversely across a plurality of tubes of varying ICG dye concentrations as represented by the motion arrow 88. The movement was imparted through a d.c. motor driven apparatus.

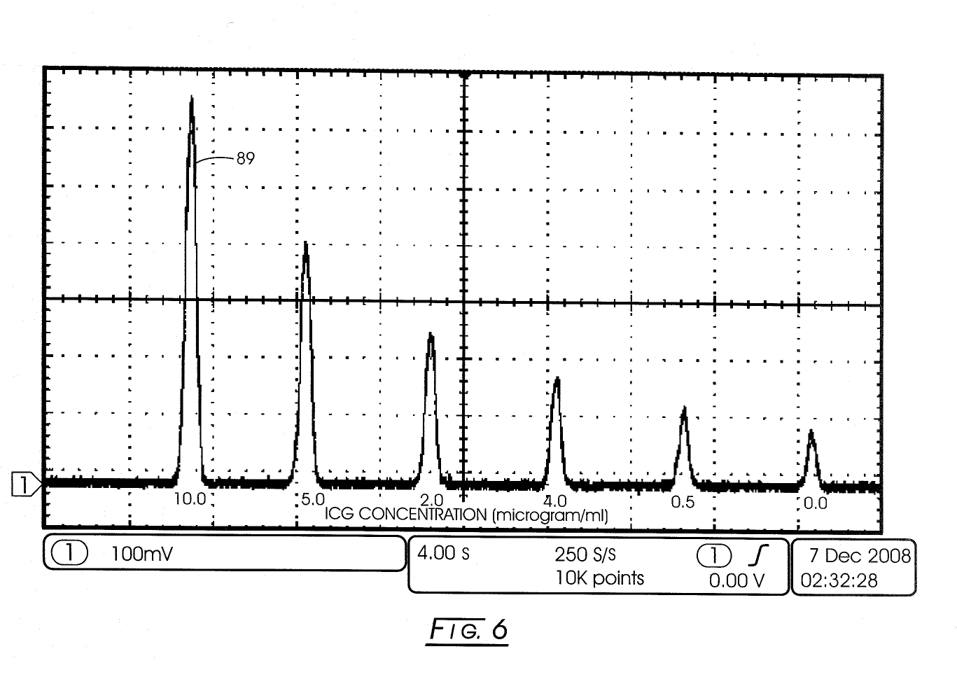

As shown in FIG. 6, a digital oscilloscope was utilized to monitor a static test carried out utilizing a plurality of sample cylinders as at 84, each carrying a unique and different ICG concentration in micrograms/milliliter (μg/ml). This test was performed in air as opposed to utilizing the skin phantom. Curve 89 of FIG. 6 demonstrates the fluorescence emissions of samples of a range of ICG concentration. Note that if the concentration of ICG elevates from 0.5 to 10.0 μg/ml, the strength of the fluorescence signal increases dramatically. Noise is represented at the 0.0 μg/ml position.

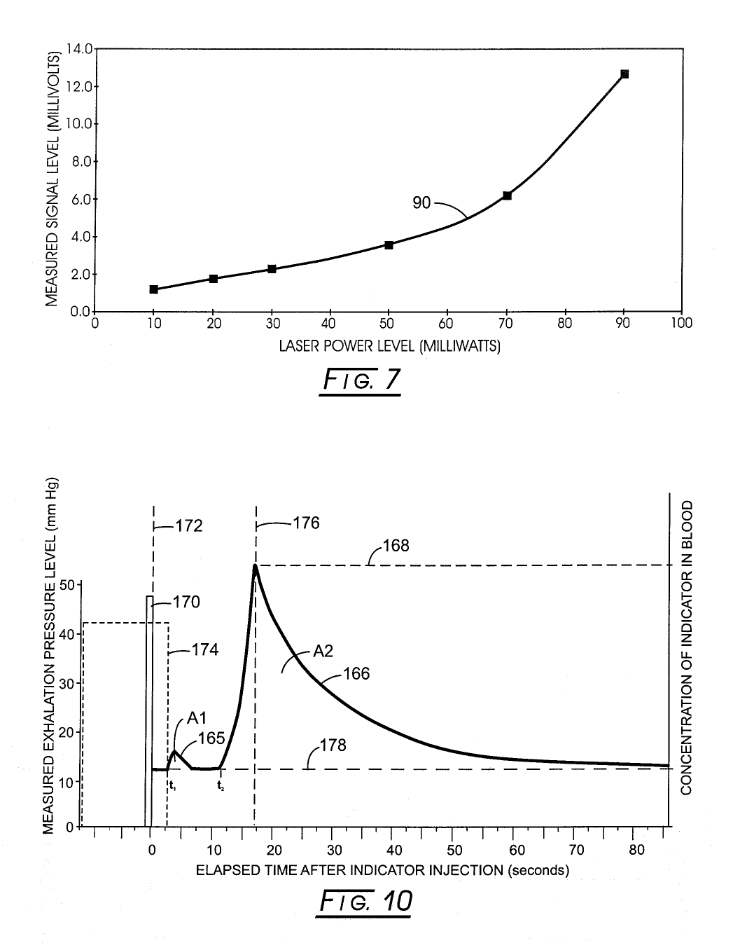

Bench top testing also evolved the information set out at curve 90 in FIG. 7. Curve 90 represents a static test in which sample vials of ICG having a concentration of 2 micrograms per ml were subjected to varying power levels (milliwatts) of an excitation laser. Note that very high signal levels in millivolts were recognized as the power level reached about 70 milliwatts.

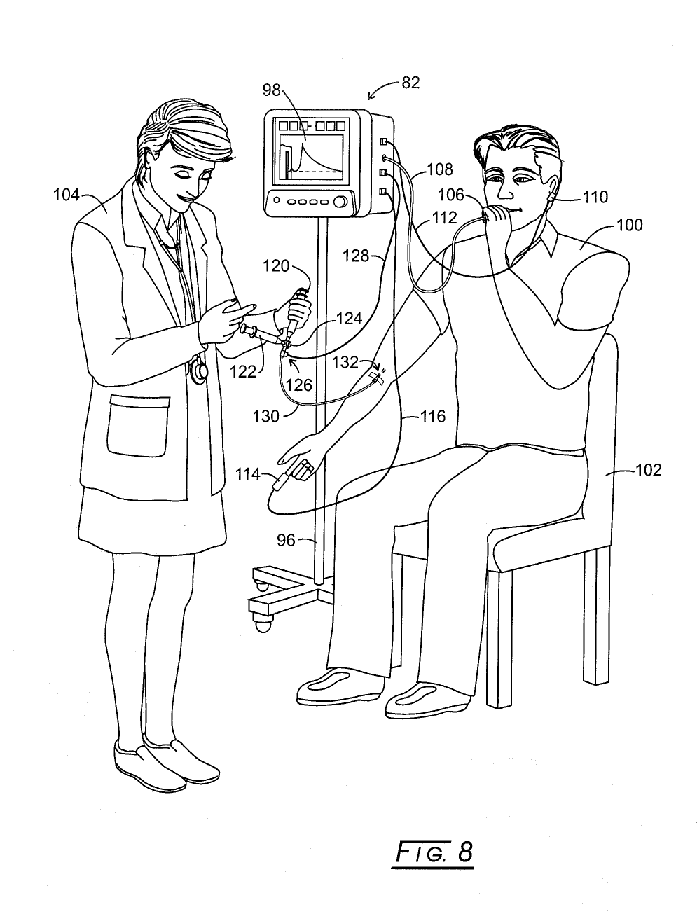

As the study is continued, researchers were able to envision the goal of providing a capability for simply and quickly screening for right-to-left cardio-cardiac shunts. Referring to FIG. 8, a rendition of the system achieving that goal is provided. In the figure, the monitor/controller/data acquisition system described at block 82 reappears with that device positioned upon a portable stand and providing, inter alia, a display represented generally at 98 having interactive features as well as showing a dye dilution type curve which may be analyzed for cardiac hemodynamics. A patient being screened is shown at 100 sitting on a chair 102. A nurse or clinician is shown at 104 administering the test under cueing guidance from the logic within device 82. Patient 100 is shown exhaling or blowing into a mouth-piece 106 which, in turn, extends via a delivery tube 108 to a manometer within the monitor function 82. The hemodynamic testing according to the present system and method in certain circumstances will preferably employ the “Valsalva Manuever,” wherein the patient 100 blows into mouth-piece 106, creating backpressure within the lungs of the patient. The Valsalva Maneuver simulates conditions under which transient cardio-cardiac shunts, e.g., atrial shunts such as a PFO, open and allow cross-flow of blood between the arterial and venous blood circulation due to the induction of a positive pressure difference between the right and left side of the heart.

As maintenance of lung back-pressure is preferable for effectively identifying shunts, monitor 82 will terminate the test during the Valsalva Maneuver if pressure is seen to drop below 40 mm of mercury. A small sensor 110 is shown attached the ear of patient 100. Sensor 110, in a preferred embodiment, incorporates three paired laser excitation components (emitters) and filtered fluorescence pick up (detector) components. These are polled to find the strongest output signal of fluorescence, such control being provided by the cable 112 operating in conjunction with the controller function at 82. (The sensor 110 is preferably positioned on the surface of the skin at the pinna of the ear). Additionally, during a period of about 60 seconds following the injection of the indicator, the patients heart rate is monitored using a conventional non-invasive heart rate monitor such as that marketed by Nellcor Pulse Oximeter, Inc. of Boulder Colo. The heart rate monitor is shown at 114 operatively associated with the monitor function 82 as represented at cable 116.

The test at hand is temporal in nature, thus proper timing of each aspect of the test is quite important. Accordingly, the control function at 82 both monitors and cues each step in the process. Note that nurse or clinician 144 is working with two syringes shown at 120 and 122. Syringes 120 and 122 are connected to a hand actuated three-way valve shown generally at 124. Syringe 120 holds a predetermined amount of indicator, while syringe 122 holds isotonic saline. The injection commencement and interval is monitored by a flow sensor represented generally at 126. From the flow sensor 126, a delivery tube 130 extends to a relatively short vein access catheter represented generally at 132. Catheter 132 is placed in a peripheral vein i.e., the antecubital vein in the arm of patient 100. Patient 100 is instructed to commence the Valsalva Maneuver thereafter maintaining a minimum of 40 mm of mercury pressure (e.g., by the patient watching the monitor display to provide visual feedback to regulate exhalation pressure level) and this effort lasting about 15 seconds will be represented as a bar on the monitor display at 98. At a cued time, injection of the indicator bolus (e.g., 2 ml of 2.5 mg/ml) indocyanine green dye over about a period of one second is immediately followed by the injection of a clearing bolus of isotonic saline (e.g., 5 ml injected over a period of 2 seconds). The rapid injection of the indicator bolus and the clearing bolus is carried out to assure that all of the injected indicator is rapidly transported from the injection site to the right atrium of the heart. If the indicator and isotonic saline are not injected within a predefined period, for example, 5 seconds before the end of the Valsalva Maneuver, then a warning audible cue is produced by the monitor function at 82.

Once the injection of the indicator bolus and isotonic saline clearing bolus have been successfully completed in the prescribed time period, the sensor detects the relative concentration of indicator. Digital filtering of the output signal from the fluorescence detectors is performed using, for example, Finite Impulse Response Digital Filtering (see Lyons, R. G., “Understanding digital signal processing”, Addison-Wellsley Publishing Company, Reading, Mass. 1997:157-204). The monitoring function at 82 tracks the passage of the indicator bolus from the peripheral vein where injected, through the right atrium and pulmonary circuit to the left atrium and ventricle and subsequent passage to arterial blood. If any part of the indicator bolus diverges from the typical blood flow pathway, for instance by a right-to-left atrial shunt, then the system is designed to detect and quantify such aberrations. For instance, if a premature increase in the indicator concentration occurs prior to the main indicator/dilution curve associated with that much larger portion of the injected indicator which takes the longer and more time consuming pathway through the lungs, then the premature increase indicates the presence of a right-to-left shunt in the heart.

In contrast to typical mammalian cardiovascular flow systems involving a single flow pathway between the injection site and the exit of the heart, a method for calculating the total flow rate of a system involving two or more individual flow pathways is described below. These abnormal cardiovascular flow systems comprise two or more indicator/dilution curves corresponding to two or more flow pathways determined at two or more physical locations corresponding to two or more separate pathways. The areas under (1) the premature smaller indication/dilution curve and (2) the much larger indicator/dilution curve can be used to ratiometrically quantify the magnitude of the right-to-left shunt.

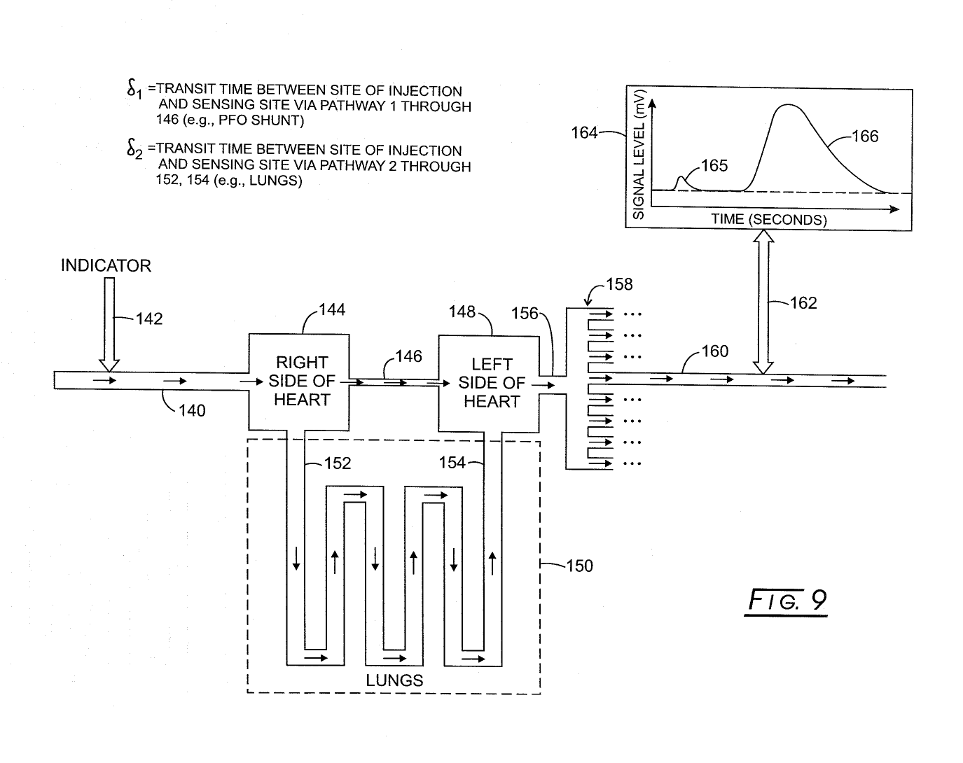

This indicator bolus premature condition and delay by spacing through the lungs can be represented schematically. Looking to FIG. 9, indicator is shown being introduced to the venous blood stream 140 as represented at arrow 142. The indicator in venous blood at blood stream 140 is directed to the right side of the heart as represented at block 144. A right-to-left shunt is represented by the small conduit 146 which is shown extending to the left side of the heart represented at block 148. Meanwhile, the lungs are represented within the dashed boundary 150, a circuitous route of filtering and aeration being represented by conduit 152 as it extends from the right side of the heart to conduit 154. As it extends from the right side of the heart following filtering and aeration as represented at conduit portion 154, the refreshed blood now enters the left side of the heart, whereupon it is distributed as represented at conduit 156 and multiple arterial conduits represented in general at 158. One conduit of the conduit array 158 is seen at 160 being analyzed by the sensor and controller function earlier described at 82 and now represented by arrow 162. Dye dilution curves are the result as shown at display 164. The principle of the dilution curves at display 164 is that the detection of an indicator bolus resulting from the passage of indicators through the lungs is seen at 166. However, note the premature and smaller indicator detection peak/dilution curve 165 which results from the passage of indicator along the shunt 146. Curve 165, representing, for example, a PFO can be quantified by a ratiometric analysis with reference to dilution curve 166. Thus, not only is the presence of a PFO detected but it is quantified. Additionally, any recirculation component will have been removed from the principal curves as at 166.

Looking momentarily to FIG. 10, curve 166 reappears with its peak concentration represented at dashed line 168. The interval of injection of the indicator bolus is represented as a bar 170, the termination of injection being represented at dashed line 172. The interval of carrying out the Valsalva Maneuver is represented by dashed block 174. The timing of system following indicator injection, in seconds, commences with dashed line 172. The elapsed time until the peak of curve 166 occurs as represented at dashed line 176 and the base line of the system (noise) is represented by horizontal dashed line 178. The passage of a portion of the indicator bolus through a shunt, such as represented at 146 of FIG. 9, is revealed by the leading curve 165, preceding curve 168 with an onset time, t₁.

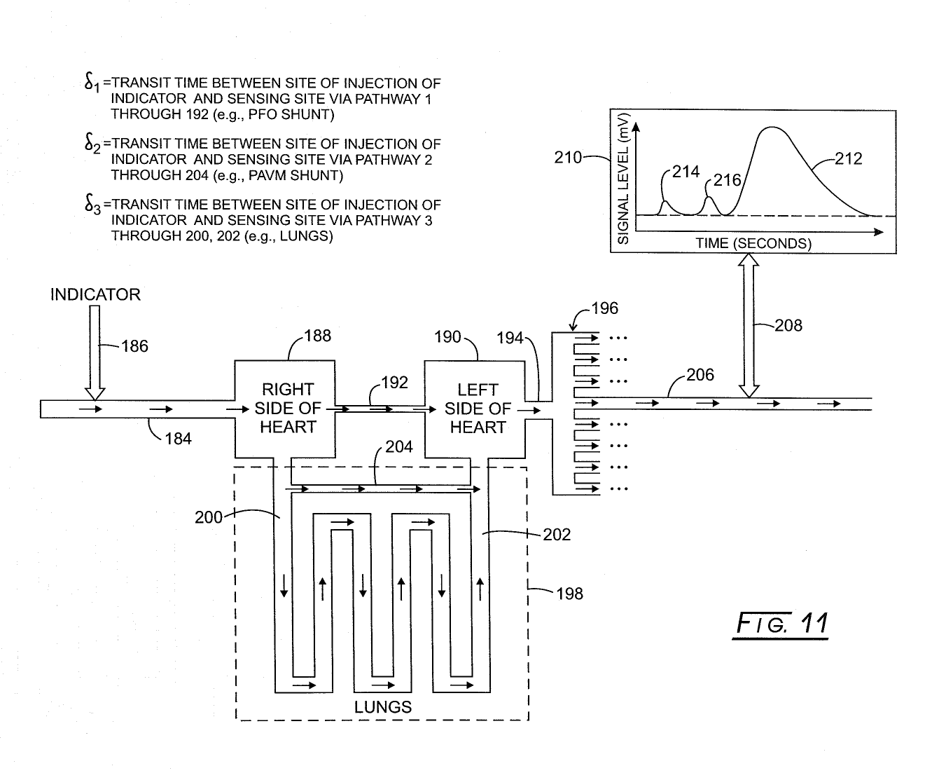

It is also possible for the pulmonary system to exhibit both a PFO and a pulmonary arterial venous malformation (PAVM), The ability to distinguish between a PFO and a PAVM is important since the closure device and method for these two types of shunts is distinctly different. Referring to FIG. 11, a combination of these conditions is schematically portrayed. As is the case of FIG. 9, circulating venous blood is represented at conduit 184 receiving indicator as represented at arrow 186. That venous blood is directed to the right side of the heart as represented at block 188. As described earlier in FIG. 9, a right-to-left shunt occurs from the right atrium of the heart to the left atrium of the heart represented at block 190. The PFO shunt being represented by conduit 192. From the left side of the heart indicator bolus is represented as passing to conduit 194 and a conduit array represented in general at 196. The lungs are represented at dashed boundary 198. Note that venous blood is shown to extend along introductory conduit 200 while filtered blood returns to the left side of the heart as represented at conduit 202. Note, however, a second right-to-left shunt as represented at conduit 204 within the pulmonary system. Conduit 204 may, for example, also represent a PAVM from the blood stream arterial conduit array 196. Another conduit is shown extending from arterial array 196 at 206. Indicator within a conduit as at 206 is subjected to excitation illumination and responds to resultant fluorescence as represented at arrow 206 and 208 and display 210. Display 210 shows a recirculation corrected principal dye dilution curve 212, a dye dilution curve 214 representing the shunt at conduit 192 and a later occurring dilution curve 216 representing the shunt described in connection with conduit 204.

Looking momentarily to FIG. 12, curves 212, 214 and 216 reappear extending above baseline concentration dashed line 220. The peak of curve 212 is represented at dashed line 222 and the interval of injection of the indicator bolus is represented at bar 224, the termination of injection being represented at vertical dashed line 226 which commences measurement of elapsed time after indicator injection (seconds). As is the case of FIG. 10, the interval of carrying out the Valsalva Maneuver is represented by dashed bar 228 and the time where the peak of curve 212 occurs is shown at dashed line 230.

### Method for Estimating Cardiac Output Using Indicator/Dilution Method

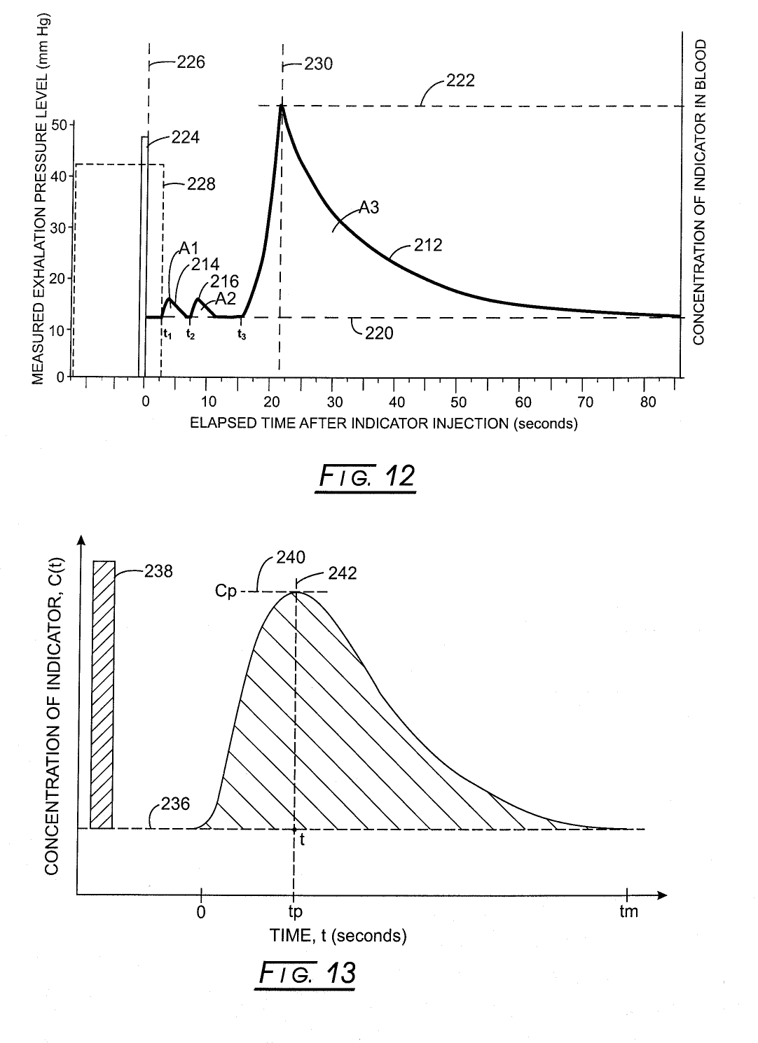

The indicator-dilution method for measuring Cardiac Output was originally developed by Hamilton during the 1920's for use with dyes as the indicator. This work by Hamilton was based on the earlier work by Stewart in 1897 and led to the following equation for calculation of the Cardiac Output of the heart, COₜ:

$$C {O}_{t} = \frac{D}{{\int }_{0}^{tm} C ( t ) d t} \qquad ( Eq . 1 )$$

where
D is the amount of indicator (e.g., grams)
C(t) is the measured concentration of the indicator at any time t (e.g., grams/ml)
∫₀^{tm} C(t) dt represents the area under the concentration vs. time curve as seen in FIG. 13 (e.g., gram-second/ml).

An exemplary indicator/dilution curve is illustrated in FIG. 13. In the example shown in FIG. 13, there is no recirculation of the indicator during the time period of the indicator/dilution from 0 to time tₘ. In this idealized case, the decay of the concentration curve following the peak at time tₚ is an exponential function once the decay curve is less than 0.70 of the peak concentration value, Cₚ. In the figure, baseline concentration is represented at dashed line 236. Indicator injection is shown at bar 238. Concentration peak is shown at dashed line 240 at time t, (dashed line 242).

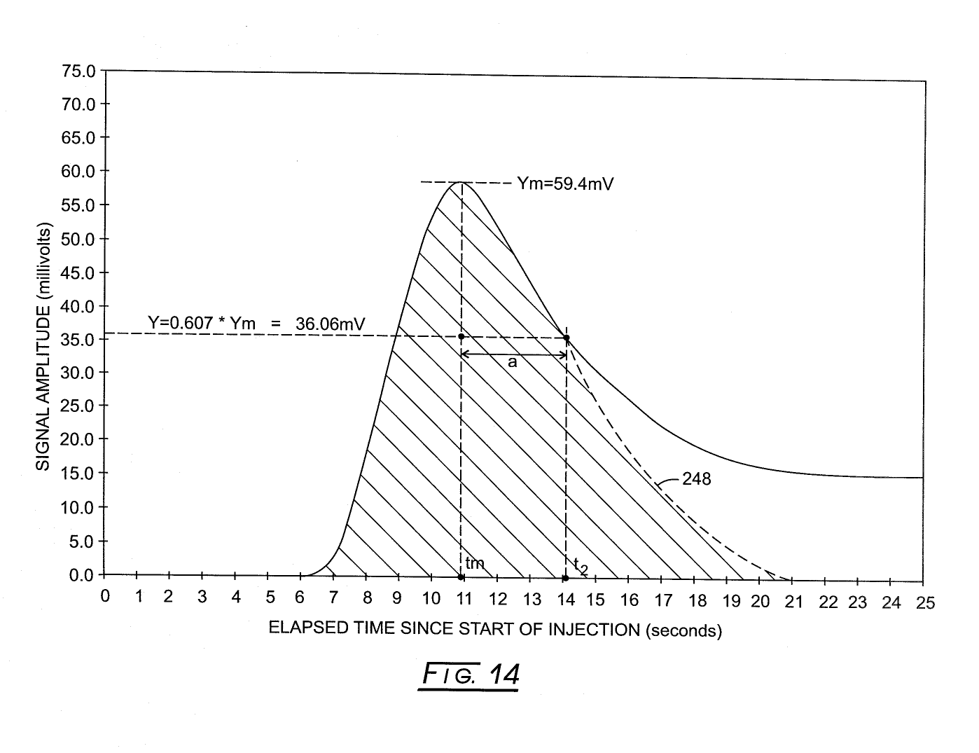

The measured indicator/dilution curve for an adult pig is shown in FIG. 14 which illustrates the effect of recirculation of a portion of the indicator back through the heart and returning to the measurement site. In the example shown in FIG. 14, the indicator, indocyanine green (ICG) dye, was injected into the left ventricle of a Yorkshire pig. The concentration of the ICG dye was measured transcutaneously at the skin surface of the ear of the pig. The relative concentration level was measured by irradiating the tissue with light at a precise wavelength of 785 nm at a power level of 100 milliwatts through a 600 micron diameter optical fiber in contact with the skin surface of the ear of the pig. The 785 nm light is directed to one or more blood vessels (preferably arterial) underlying the skin surface. As the ICG indicator within the blood stream reaches the site of irradiation with 785 nm light, the fluorescent component within the ICG indicator is excited to an elevated energy state for a brief period. As the excited component returns to its normal energy state, it emits light at a longer wavelength (viz., 830 nm) and the difference between the excitation wavelength (viz., 785 nm) and the fluorescence emission wavelength (viz., 830 nm) is known as the Stokes Shift. This Stokes Shift of nominally 45 nm allows the fluorescence emission to be extracted by using an interferential or other type of optical filter to reflect or otherwise attenuate all but the wavelength band of interest (viz., 820 to 840 nm). The fluorescence emissions collected in this example by seven 600 micron diameter optical fibers surrounding the excitation fiber (see FIG. 3) to be subjected to an optical filter which causes a significant attenuation of all light wavelengths except those in the near vicinity of the fluorescence wavelength of interest, viz., 830 nm. In this manner, the excitation light at 785 nm is blocked by the optical filter.

In addition to optical filtering, Fast Fourier Transform filtering methods or Finite Impulse Response (nonrecursive) filtering methods can be used to remove spectral content from the measured fluorescence signal which is outside the frequency band of interest. In the present example, the digital filtering of the fluorescence signal serves as a “low pass” filter allowing only signals with a frequency component in the range from 0 to 0.8 Hz to be included in the digitally filtered form of the measured data.

Referring again to FIG. 14, several methods can be used to exclude the recirculation component of the indicator/dilution curve so that the area under the idealized indicator/dilution curve can be obtained, i.e., the indicator/dilution curve without the recirculation component. One method is illustrated in FIG. 14 and involves the assumption that the down slope of the indicator/dilution curve is a log-normal distribution.

For further discussion of measuring cardiac output and calculating the area under the dye dilution curve, see:
13) Moore, J., Kinsman, J., Hamilton, W. and Spurling, R., “Studies on the Circulation 2. Cardiac Output Determinations: comparison of the Injection Method with the Direct Fick Procedure,” American Journal of Physiology 89: 331-339 (1929);
14) Snoeckx L., et al., “On-Line Computation of Cardiac Output with the Thermodilution Method using a Digital Minicomputer,” Cardiovascular Research, 10: 556-564, (1976);
15) Lewi P., “Areas under thermal-dilution curves, assuming log-normal distribution,” American Journal of Physiology, 207(1): 144-148 (1964).

Utilizing the presently derived method, the area under the indicator/dilution curve with the recirculation component removed, A can be calculated as follows:

A=Yₘ*a*c (Eq. 2)

where
A=area under indicator/dilution curve without recirculation component (millivolt-seconds)
Yₘ=maximum signal amplitude (millivolts)
a=width of indicator/dilution curve to right of time, tₘ at Y=0.607*Yₘ (seconds)
c=constant value=2.20

As seen in FIG. 14, the above equation estimates the area under the indicator/dilution curve shown by the dashed line 248 which represents the shape of the down slope of the indicator/dilution curve without recirculation.

Another method which can be used to estimate the shape of the down slope curve without the recirculation component involves the use of least mean squares regression for the exponential curve fit to obtain the coefficient, α in the equation.

Y=Y₂e^{-∝(t-t}^{₂}⁾ (Eq. 3)

where
Y is the signal amplitude at time, t (millivolts)
Y₂ is the signal amplitude at time, t₂ (millivolts)
t-t₂ is the elapsed time following time t₂ (seconds)
α is the exponential coefficient which defines the signal amplitude down-slope curve and is derived by an exponential curve fit to the actual measured signal amplitude values in time interval between t₁ and t₂
t₁ is the time at which Y₁=F₁*Yₘ
t₂ is the time at which Y₂=F₂*Yₘ
F₁ and F₂=0.9 and 0.6, respectively

### Method for Measuring Shunt Conductance

Referring again to Eq. 1, note that the integral in the denominator of the expression for Cardiac Output, COₜ is in terms of a concentration value, C(t) and it is in the same unit as the amount of the indicator injected, D. By way of example, the amount of indicator injected, D is in units of micrograms and the concentration of the indicator, C(t) is in units of microgram/milliliter or microgram/ml. However, the presently disclosed non-invasive method for measuring the Shunt Conductance, C involves the measurement of a relative concentration value which, by way of example, is in unit of millivolts. Therefore, Eq. 1 can be rewritten in terms of measurement of a signal amplitude, V(t) as a function of time to obtain a volumetric flow rate as follows:

$$C {O}_{t} = \frac{D}{C C F * {\int }_{0}^{t} V ( t ) d t} \qquad ( Eq . 4 )$$

where
COₜ=volumetric flow rate (milliliter/second)
D=amount of indicator injected (micrograms)
V(t)=measured signal level as a function of time which is directly proportional to the concentration of the indicator in the blood stream (millivolts)
CCF=indicator concentration conversion factor to convert measured signal level (in millivolts) into indicator concentration (micrograms/ml) and which has the unit (micrograms/ml-millivolt).

The indicator concentration conversion factor, CCF is a constant value for a specific set of transcutaneous measurement fluorescence measurement parameters which include (1) excitation light intensity (e.g., laser power in units of milliwatts), (2) thickness of skin between surface of light source/fluorescence detector and blood vessel(s) carrying blood-borne indicator, (3) quantum efficiency of the fluorescing indicator or indicators (4) light scattering characteristics of the skin and skin type, (5) amplifier gain in fluorescence detection circuit and (6) attenuation of fluorescence light flux through optical filtering component(s).

As discussed above related to the calculation of Cardiac Output using indicator/dilution methods, the integral term corresponds to the area under the curve defined by the measured signal level as a function of time. Also, as seen before in FIG. 14, the recirculation effects must be excluded by extrapolating an exponential down slope curve from measure data measured briefly after the time at which the peak signal level is measured. Rewriting Eq. 4 in terms of the area under the curve, A, after recirculation components have been excluded provides:

$$C {O}_{t} = \frac{D}{C C F * A} \qquad ( Eq . 5 )$$

The new method described is useful for detecting the presence of a right-to-left shunt as has been shown in FIG. 9 and FIG. 10. If a right-to-left shunt is present then a relatively small indicator/dilution curve will precede the much larger indicator/dilution curve. The former relatively small indicator/dilution curve with area, A₁, is associated with that amount of the blood-borne indicator originally injected, D which passes through a right-to-left shunt without first passing through the longer and more time-consuming pathway through the lungs. The latter, much larger indicator/dilution curve with area, A₂ is associated with that amount of the blood-borne indicator originally injected, D which flows along the normal pathway from the right atrium to the right ventricle and through the lungs to then reach the left atrium and proceeding on to the arterial circulation. This latter pathway requires a longer transit time which results in a starting time for this latter curve, t₂ associated with the normal blood flow pathway occurring later than the starting time for the former curve, t₁ associated with the abnormal right-to-left shunt flow pathway as illustrated at 146 in FIG. 9.

### Calculating Individual Flow Rates for Two or More Indicator/Dilution Curves Separated in Time

The calculation the volumetric flow rate in Eqs. 1, 4 and 5 corresponds to a mammalian circulatory system in which there is only one pathway for the flow of liquid (e.g., blood) and injected indicator from the site of indicator injection (e.g., peripheral vein) to the ascending aorta exiting the left ventricle of the heart. Alternatively, two or more indicator/dilution curves result if there are two or more flow pathways having different transit times within the system which then converge at a single measurement site at a location downstream from the two or more pathways. By way of example, such a system with two pathways in which pathway 146 has a shorter transit time δt₁ and pathway 152-154 has a longer transit time, δt₂ is shown in FIG. 9 where transit time, δt₁ refers to elapsed time from the time of indicator injection to the time of the onset of the initial rise in indicator/dilution curve.

As illustrated in FIG. 9 at 164, two distinct indicator/dilution curves can be measured at a single downstream site where the indicator/dilution curves are separated in time by the difference between the transit times δ₁ and δ₂. As before, the total flow rate, COₜ for this flow system involving two flow components can be calculated.

As seen in this hypothetical flow system illustrated in FIG. 9, the flow rate for the total system, COₜ can be calculated by combining the areas under each of the two indicator/dilution curves 165 and 166 in each of the two pathways for the atypical flow of liquid which can be calculated based on Eq. 5 as follows:

$$C {O}_{t} = \frac{D}{C C F * {A}_{t}} = \frac{D}{C C F * ( {A}_{1} + {A}_{2} )} \qquad ( Eq . 6 )$$

where
COₜ=volumetric flow rate (e.g., milliliter/second)
D=amount of indicator injected (e.g. micrograms)
CCF=indicator concentration conversion factor to convert measured signal level (e.g. millivolts) into indicator concentration (e.g., micrograms/ml-millivolts)
Aₜ=total area under one or more indicator/dilution curves corresponding to one or more pathways for the flow of liquid in the system (e.g., millivolts-seconds)
A₁=area under the indicator/dilution curve 165 for flow pathway 146 (e.g., millivolt -second)
A₂=area under the indicator/dilution curve 166 for flow pathway 152-154 (e.g., millivolt-second)

As illustrated in FIG. 9 in a liquid flow system where two flow pathways result in two indicator/dilution curves, the flow rate in pathway 1, C₁ can be calculated ratiometrically as follows:

$$C {O}_{1} = C {O}_{t} * \frac{{A}_{1}}{{A}_{1} + {A}_{2}} \qquad ( Eq . 7 )$$

where
CO₁=volumetric flow rate in pathway 146 (e.g., milliliter/second)
COₜ=total volumetric flow rate for both pathways 146 and 154 (e.g., milliliter/second)
A₁=area under the indicator/dilution curve 165 for flow through pathway 146 (e.g., millivolt-second)
A₂=area under the indicator/dilution curve 166 for flow through pathway 152-154 (e.g., millivolt-second)

For the case of equal volumetric flow rates in pathways 146 and 152-154, the areas under the curves, A₁ and A₂ will be equal. In this exemplary case, Eq. 7 can be written as follows (where A₁=A₂):

$$C {O}_{1} = C {O}_{t} \frac{{A}_{1}}{2 {A}_{1}} = \frac{1}{2} * C {O}_{t} \qquad ( Eq . 8 )$$

Alternatively, if the volumetric flow rate at pathway 146 is only one-tenth of the flow rate through pathway 154, then A₂=10*A₁ and Eq. 7 can be written in this exemplary case as follows (where A₂=10*A₁):

$$C {O}_{1} = C {O}_{t} \frac{{A}_{1}}{{A}_{1} + 10 {A}_{1}} = \frac{1}{11} * C {O}_{t} \qquad ( Eq . 9 )$$

As illustrated in FIG. 11 in a case of a flow system with three pathways in which pathway 192 has the shortest transit time, δt₁ while pathway 204 has a longer transit time, δt₂ and pathway 202 has the longest transit time, δt₃ is shown in FIG. 12. As illustrated in FIG. 12, three distinct indicator/dilution curves can be measured at a single downstream site where the three indicator/dilution curves are separated in time by the differences between the transit times, δt₁, δt₂ and δt₃. The total flow rate, COₜ for this flow system involving three flow components can be calculated by combining the areas under each of the three indicator/dilution curves corresponding to each of the three pathways for the flow of liquid which can be calculated based on Eq. 5 as follows:

$$C {O}_{t} = \frac{D}{C C F * {A}_{t}} = \frac{D}{C C F * ( {A}_{1} + {A}_{2} + {A}_{3} )} \qquad ( Eq . 10 )$$

where
COₜ=volumetric flow rate (e.g., milliliter/second)
D=amount of indicator injected (e.g. micrograms)
CCF=indicator concentration conversion factor to convert measured signal level (e.g. millivolts) into indicator concentration (e.g., micrograms/ml-millivolts)
Aₜ=total area under one or more indicator/dilution curves corresponding to one or more pathways for the flow of liquid in the system (e.g., millivolts-seconds)
A₁=area under the indicator/dilution curve for flow pathway 192, curve 214 (e.g., millivolt-second)
A₂=area under the indicator/dilution curve for flow pathway 204, curve 216, (e.g., millivolt-second)
A₃=area under the indicator/dilution for the primary pathway 200-202 through lungs, curve 212 (e.g., millivolt-second)

As illustrated in FIG. 11 in a liquid flow system where three flow pathways result in three indicator/dilution curves, the flow rate for any single pathway can be calculated ratiometrically as follows for the case of flow rate in pathway 192:

$$C {O}_{1} = C {O}_{t} * \frac{{A}_{1}}{{A}_{1} + {A}_{2} + {A}_{3}} \qquad ( Eq . 11 )$$

where
CO₁=volumetric flow rate in pathway 1 (e.g., milliliter/second)
COₜ=total volumetric flow rate for both pathways 1 and 2 (e.g., milliliter/second)
A₁=area under the indicator/dilution curve for flow through pathway 192 (e.g., millivolt-second)
A₂=area under the indicator/dilution curve for flow through pathway 204 (e.g., millivolt-second)
A₃=area under the indicator/dilution curve for flow through the main pathway 200-202 through lungs (e.g., millivolt-second)

For the case of equal volumetric flow rates in the three pathways, the areas under the curves A₁, A₂ and A₃ will be equal based on the principles of the Steward-Hamilton equation for indicator/dilution based flow rate measurement as discussed with regard to Eqs. 4 and 5. Hence, Eq. 11 can be written in this exemplary case as follows (where A₁=A₂=A₃):

$$C {O}_{1} = C {O}_{t} * \frac{{A}_{1}}{3 {A}_{1}} = \frac{1}{3} * C {O}_{t} \qquad ( Eq . 12 )$$

For the case of a flow system with two flow pathways the flow rate in any single pathway, COᵢ is giving by the general relationship:

$$C {O}_{i} = C {O}_{t}^{*} ( \frac{{A}_{i}}{{A}_{1} + {A}_{2}} ) \qquad ( Eq . 13 )$$

where
COᵢ=volumetric flow rate in pathway i (e.g., milliliter/second)
COₜ=total volumetric flow rate for both pathways 1 and 2 (e.g., milliliter/second)
Aᵢ=area under the indicator/dilution curve for flow through pathway i (e.g., millivolt-second)
A₁=area under the indicator/dilution curve for flow through pathway 192 (e.g., millivolt-second)
A₂=area under the indicator/dilution curve for flow through pathway 204 (e.g., millivolt-second)
i=pathways 192 or 204
   For the case of a flow system with three flow pathways as illustrated in FIG. 11, the flow rate in any single pathway, COᵢ is giving by the general relationship:

$$C {O}_{i} = C {O}_{t}^{*} ( \frac{{A}_{i}}{{A}_{1} + {A}_{2} + {A}_{3}} ) \qquad ( Eq . 14 )$$

where
COᵢ=volumetric flow rate in pathway i (e.g., milliliter/second)
COₜ=total volumetric flow rate for all pathways (e.g., milliliter/second)
Aᵢ=area under the indicator/dilution curve for flow through pathway i (e.g., millivolt-second)
A₁=area under the indicator/dilution curve for flow through pathway 192 (e.g., millivolt-second)
A₂=area under the indicator/dilution curve for flow through pathway 204 (e.g., millivolt-second)
A₃=area under the principal indicator/dilution (e.g., millivolt-second)
i=one of the three pathways

### Identifying the Presence of and Quantifying the Flow Rate Across Right-to-Left Shunts in the Human Body

The case of a flow system with two flow pathways exists in the human body when a right-to-left shunt is present in the heart or the pulmonary circulation. As described above, the most common form of a right-to-left shunt in the heart is known as a Patent Foramen Ovale or PFO. A method apparatus and system are next described which determines the magnitude of the flow rate associated with a right-to-left shunt in the heart and/or within the pulmonary vasculature. After positioning, a sensor, for instance, an optical fluorescence sensor on the surface of the subject, the circulatory tracking reagent is injected at a predetermined rate into a peripheral vein of the subject while the subject exhales into a manometer mouthpiece. The exhalation by the patient into a pressure-sensing mouthpiece to a pressure of at least 40 mmHg is preferred for producing the pressure differential necessary to cause blood to flow across a right-to-left shunt such as a PFO. The Valsalva maneuver may cause a patent foramen ovale, if present, to open, allowing blood to flow directly from the right atrium to the left atrium of the heart without passing through the longer pathway in the lungs. The optical sensor transcutaneously measures the concentration of the injected indicator as a function of time. As seen in FIG. 10, if a premature inflection or peak occurs in the indicator concentration level at a time point, t₁ prior to the rise and fall of the concentration associated with the majority of the indicator flowing through the normal pathway of the lungs and arriving at the sensor at time point t₂, then a right-to-left shunt (e.g., PFO) is present in the heart. The situation in the human body where only one right-to-left shunt exists is illustrated schematically in FIG. 9 in which two flow pathways exist having different transit times δt₁ and δt₂.

Alternatively, there may be two types of right-to-left shunt in the human body which represent two additional pathways for the flow of blood as illustrated in FIG. 11. As stated above, if a sensor is used to measure the concentration of the injected indicator as a function of time at a location downstream from the left side of the heart (e.g., the auricle of the ear), then a total of three indicator/dilution curves result as seen in FIG. 12. Still referring to FIG. 12, the first indicator/dilution curve, having an area, A₁ under this curve, is associated with the right-to-left shunt having the shortest pathway between the right atrium and the left atrium of the heart. An example of such a shorter pathway is a pathway directly across the atrial septum such as a PFO. The second indicator/dilution curve, having an area, A₂ under the curve, is associated with a right-to-left shunt having a longer pathway between the right atrium and the left atrium of the heart. An example of such a longer pathway is a particular arteriovenous malformation creating a pathway across the pulmonary vasculature commonly known as a Pulmonary Arteriovenous Malformation (PAVM). In the cases where there are either one or two right-to-left shunts, a much larger indicator/dilution curve also occurs which is associated with that amount of blood which normally flows through the much longer pathway in the lungs and back to the left atrium of the heart. This larger indicator/dilution curve is seen in FIG. 10 having an area under the curve (after excluding any recirculation component as described previously) of A₂ or in FIG. 12 having an area under the curve (after excluding any recirculation component as described previously) of A₃.

For the case of a human subject with one right-to-left shunt detected as seen in FIG. 10, the magnitude of the flow rate associated with the right-to-left shunt, referred to as “Shunt Conductance,” is given by Eq. 13 as follows:

$$Shunt Conductance for single right \text{-} to \text{-} left shunt = C {O}_{1} = C {O}_{t} * \frac{{A}_{1}}{{A}_{1} + {A}_{2}} \qquad ( Eq . 15 )$$

where the terms are the same as those defined for Eqs. 7 and 13.

Likewise, for the case of a human subject with two right-to-left shunts detected as seen in FIG. 12, the magnitude of the flow rate associated with each of the right-to-left shunts, referred to as “Shunt Conductance,” is given by Eq. 14 as follows:

$$\begin{matrix}Shunt Conductance for first right \text{-} to \text{-} left shunt = C {O}_{1} = C {O}_{t} * \frac{{A}_{1}}{{A}_{1} + {A}_{2} + {A}_{3}} & ( Eq . 16 ) \\ Shunt Conductance for second right \text{-} to \text{-} left shunt = C {O}_{2} = C {O}_{t} * \frac{{A}_{2}}{{A}_{1} + {A}_{2} + {A}_{3}} & ( Eq . 17 )\end{matrix}$$

where the terms are the same as those defined for Eq. 14.

As seen in Eqs. 15, 16 and 17, the areas A₁, A₂ and A₃ are calculated parameters based on the measurement of indicator concentration as a function of time for each of the indicator/dilution curves that corresponds to the presence of a single right-to-left shunt (see A₁ in FIG. 10) or two right-to-left shunts (see A₁ and A₂ in FIG. 12). In the preferred embodiment of the present invention, indicator/dilution curves are measured in terms of a fluorescence signal level (e.g., millivolts) as a function of time. The concentration conversion factor, CCF appearing in Eqs. 6 and 10 is cancelled out since it appears in both the numerator and denominator of Eqs. 7, 11, 13 and 14.

As seen in Eq. 7 and FIG. 10 for the case of a single right-to-left shunt, the total volumetric flow rate for both pathways, COₜ is equivalent to the Cardiac Output of the heart. The value for the Cardiac Output can be calculated for the case of a human under normal conditions (e.g., a human which is not concurrently undergoing surgery, post-surgery, recovery from anesthesia, or suffering from shock due to heart failure of trauma-related blood loss). Importantly, subjects to be screened for the presence of one or more right-to-left shunts using the present invention will be tested when their cardiovascular condition can be assumed to be normal. Likewise, the evaluation of the presence and flow rate associated with any residual right-to-left shunt following closure of the shunt will also be performed on human subjects whose cardiovascular condition can be assumed to be normal at the time of the test. For the case of an adult human, the normal value of the Stroke Index, SI is known to be 46±5 ml/m² (see Shoemaker, W. C., et al., “Textbook of Critical Care,” W.B. Saunders Company, Philadelphia 1995: pg. 263). As seen in the Shoemaker reference, Stroke Index, SI is related to Cardiac Index, CI and heart rate, HR as follows:

$$S I = \frac{C I}{H R} \qquad ( Eq . 18 )$$

where
SI=Stroke Index (ml/m²)
CI=Cardiac Index (ml/second-m²)
HR=heart rate (beats per second) determined by measuring beats per minute and dividing by 60 seconds/minute

As seen in the cited Shoemaker reference, the Cardiac Index, CI is equal to the Cardiac Output, CO divided by the Body Surface Area, BSA as follows:

$$C I = \frac{C O}{B S A} \qquad ( Eq . 19 )$$

where
CI=Cardiac Index (ml/m²)
CO=Cardiac Output (ml/second)
BSA=Body Surface Area (square meters or m²)

The Body Surface Area (BSA) can be calculated by one of several algorithms incorporating the height and weight of the human subject. One of the recommended algorithms used to calculate the Body Surface Area, BSA is the algorithm known as the Mosteller Formula (see Mosteller, R. D., “Simplified Calculation of Body Surface Area,” New England Journal of Medicine 1987; 17[17]: 1098). The Mosteller Formula for Body Surface Area is given by the following equation:

BSA=((H*W)/3131)^{1/2} (Eq. 20)

where
BSA=Body Surface Area (m²)
H=Height of subject (inches)
W=Weight of subject (pounds)

The normal value of the Cardiac Output for a human subject can then be calculated by solving Eqs. 18 and 19 for Cardiac Output, CO as follows. First, rewriting Eq. 19 in terms of Cardiac Output, CO obtains:

CO=CI*BSA (Eq. 21)

Rewriting Eq. 18 in terms of Cardiac Index, CI obtains:

CI=SI*HR (Eq. 22)

Substituting formula for Cardiac Index, CI (Eq. 22) into formula for Cardiac Output, CO in Eq. 21 obtains:

CO=CI*BSA=SI*HR*BSA (Eq. 23)

where:
a. the normal value for SI is known to be 46±5 ml/m²
b. HR is measured using a heart rate monitor and averaged over the period of the measurement of the relative concentration of the indicator during the period from the indicator injection to the time of the peak of the normal indicator/dilution curve associated with blood flow through the lungs and back to the left atrium of the heart (beats/second)
c. BSA is calculated using Mosteller Formula given in Eq. 20 in units of m²

Hence, the normal value of the total Cardiac Output can be estimated within a narrow range based on the known normal value for Stroke Index, SI (viz., 46±5 ml/m²), by measuring the subjects heart rate, HR and calculating the Body Surface Area, BSA based on the subjects height and weight. This estimated value for Cardiac Output, CO is equivalent to the total flow rate, COₜ incorporated into Eqs. 7, 11, 13 and 14. By way of example and referring to Eq. 7, Eq. 23 and FIG. 10, the flow rate or Shunt Conductance, CO₁ associated with a single right-to-left shunt can be written:

$$C {O}_{1} = C {O}_{t} * \frac{{A}_{1}}{{A}_{1} + {A}_{2}} = S I * H R * B S A * \frac{{A}_{1}}{{A}_{1} + {A}_{2}} \qquad ( Eq . 24 )$$

where:
CO₁=flow rate or Shunt Conductance through right-to-left shunt (ml/second)
SI=Stroke Index whose normal value is 46±5 ml/m²
HR=Heart Rate which is measured using a heart rate monitor (beats/second)
BSA=Body Surface Area calculated using Mosteller Formula (m²)
A₁=calculated area under measured indicator/dilution indicator concentration curve associated with right-to-left shunt as seen in FIG. 10 (millivolt-second)
A₂=calculated area under measured indicator/dilution indicator concentration curve associated with blood flow through the lungs and back to left atrium of the heart as seen in FIG. 10 (millivolt-second)

Hence, Eq. 24 allows the equivalent flow rate or Shunt Conductance through a single right-to-left shunt to be calculated within a narrow range (based on normal range of Stroke Index of 46±5 ml/m²). Likewise, Eq. 14 for the case of two co-existing right-to-left shunts can be re-written as follows:

$$\begin{matrix}\begin{matrix}C {O}_{1} = C {O}_{t} * \frac{{A}_{1}}{{A}_{1} + {A}_{2} + {A}_{3}} \\ = S I * H R * B S A * \frac{{A}_{1}}{{A}_{1} + {A}_{2} + {A}_{3}}\end{matrix} & ( Eq . 25 ) \\ \begin{matrix}C {O}_{2} = C {O}_{t} * \frac{{A}_{2}}{{A}_{1} + {A}_{2} + {A}_{3}} \\ = S I * H R * B S A * \frac{{A}_{2}}{{A}_{1} + {A}_{2} + {A}_{3}}\end{matrix} & ( Eq . 26 )\end{matrix}$$

where
CO₁=flow rate or Shunt Conductance through a first right-to-left shunt (ml/second)
CO₂=flow rate or Shunt Conductance through a second right-to-left shunt (ml/second)
SI=Stroke Index whose normal value is 46±5 ml/m²
HR=Heart Rate which is measured using a heart rate monitor (beats/second)
BSA=Body Surface Area calculated using Mosteller Formula (m²)
A₁=calculated area under a first measured indicator/dilution indicator concentration curve associated with right-to-left shunt (millivolt-second)
A₂=calculated area under a second measured indicator/dilution indicator concentration curve associated with shunt flow through pulmonary arterial pathway (e.g., PAVM) and back to left atrium of the heart (millivolt-second)
A₃=calculated area under a third measured indicator/dilution indicator concentration curve associated with blood flow through lungs and back to left atrium of heart (millivolt-second)
   Benefits of Ratiometric Analysis Method for Quantifying the Volumetric Flow Rate Associated with Right-to-Left Shunts

As a result of the ratiometric approach specified in the present invention for calculating the flow rate or Shunt Conductance associated with one or two right-to-left shunts, several terms required for the calculation of the volumetric flow rate of a system are cancelled out and therefore do to have to be determined. Note that Eq. 6 for the case of calculating the flow rate in a system with two flow pathways includes the parameters for (a) the amount of the indicator injected, D (e.g., grams) and (b) the factor for converting the measured signal level (in millivolts) into a concentration level, CCF (e.g., grams/ml).

However, due to the ratiometric form of Eq. 7 in the present invention, the terms D and CCF cancel out. Also, the units of measure for the fluorescence signal level (e.g., millivolts-seconds) cancel out. As a result, the exact amount of the indicator injected does not have to be accurately known. However, the amount of the indicator injected needs to be sufficiently large so that the much smaller indicator/dilution curves associated with one or two right-to-left shunts can be adequately resolved and their corresponding areas can be accurately calculated. In a preferred embodiment of the present invention involving the use of Indocyanine Green (ICG) dye (supplied, for example, by Akorn, Inc., 2500 Millbrook Drive, Buffalo Grove, Ill. 60089) as the indicator, the preferred amount of ICG dye injected is 2.0 ml at an ICG dye concentration of 2.5 milligram/ml which is equivalent to an injected ICG dose of 5.0 milligrams. The chemical name for Indocyanine Green is 1H-benz(e)indolium, 2-[7-[1,3-dihydro-1,1-dimethyl-3-(4-sulfobutyl)-2H-benz[e]indo-2-ylidene]-1,3,5-heptatrienyl]-1,1-dimethyl-3-(4-sulfobutyl)-hydroxide, inner salt, sodium, or 2-[7-[1,1-dimethyl-3-(4-sulfobutyl)benz[e]indolin-2-ylidene]-1,3,5-heptatrienyl]-1,1-dimethyl-3-(4-sulfobutyl)-1H-benz[e]indolium hydroxide, inner salt, sodium salt. A typical right-to-left shunt test procedure might involve a total of 3 to 5 tests which represents a total injected dose of 15 to 25 milligrams. This cumulative dosage amount is well below the manufacturers specified guideline which recommends a maximum ICG dose per day of 2 milligrams per kilogram of body weight. The recommended ICG dose for a human subject weighting 140 pounds or 63.6 kg is about 127 milligrams, a factor of 5 to 8.5× higher than the amount of ICG dye required to perform multiple right-to-left shunt measurements on a single human subject weighing 140 pounds. This significant ratio of the actual to recommended dosage limit assures that the method of the present invention can be safely employed.

### EXAMPLES

### Example 1

### Calculation of Flow Rate of Human Subject with Single Right-to-Left Shunt

This example involves the calculation of the flow rate or Shunt Conductance of a human subject with a single right-to-left shunt, as seen in FIG. 9 using Eq. 24 and assuming the following parameters:

### Assume:

SI=Stroke Index for Normal Subject=46 ml/m²
H=Height of subject=70 inches
W=Weight of subject=160 pounds
HR=measured average heart rate during period of measurement of indicator concentration as a function of time=1.0 beats/second (i.e., 60 beats/minute)
A₁=calculated area under indicator/dilution curve derived from measured fluorescence signal level (in units of millivolts) vs. time (in seconds) associated with right-to-left shunt=40.5 millivolts-seconds
A₂=calculated area under indicator/dilution curve derived from measured fluorescence signal level (in units of millivolts) vs. time (in seconds) associated with blood flow through lungs and back to left atrium of heart=3000 millivolts-seconds
   Using subject's actual height and weight, the Body Surface Area, BSA is calculated using Eq. 20 as follows:

BSA=((H*W)/3131)^{1/2}=((70 inches*160 pounds)/3131)^{1/2}=1.89 m² (Eq. 27)

Substituting the measured and calculated parameters A₁, A₂ and heart rate as well as the normal Stroke Index value into Eq. 24 provides the calculated value for the flow rate or Shunt Conductance, C₁ as follows:

$$C {O}_{1} = S I * H R * B S A * \frac{{A}_{1}}{{A}_{1} + {A}_{2}}$$

$$C {O}_{1} = ( 46 ml \text{/} {m}^{2} ) * ( 1.0 beats \text{/} sec ) * ( 1.89 {m}^{2} ) * \frac{40.5 mV \text{-} secs}{40.5 mV \text{-} secs + 3000 mV \text{-} secs}$$

$$C {O}_{1} = 1.16 ml \text{/} sec$$

### Example 2

### Calculation of Flow Rate of Human Subject with Two Right-to-Left Shunts

This example involves the calculation of the flow rate or Shunt Conductance of a human subject with two right-to-left shunts, as seen in FIG. 11, using Eq. 25 and 26 assuming the following parameters:

### Assume:

SI=Stroke Index for Normal Subject=46 ml/m²
H=Height of subject=72 inches
W=Weight of subject=185 pounds
HR=measured average heart rate during period of measurement of indicator concentration as a function of time=1.2 beats/second (i.e., 60 beats/minute)
A₁=calculated area under indicator/dilution curve derived from measured fluorescence signal level (in units of millivolts) vs. time (in seconds) associated with a first right-to-left shunt=45.0 millivolts-seconds
A₂=calculated area under indicator/dilution curve derived from measured fluorescence signal level (in units of millivolts) vs. time (in seconds) associated with right-to-left shunt=65.7 millivolts-seconds
A₃=calculated area under indicator/dilution curve derived from measured fluorescence signal level (in units of millivolts) vs. time (in seconds) associated with blood flow through lungs and back to left atrium of heart=3100 millivolts-seconds
   Using subject's actual height and weight, the Body Surface Area, BSA is calculated using Eq. 20 as follows:

BSA=((H*W)/3131)^{1/2}=((72 inches*185 pounds)/3131)^{1/2}=2.06m² (Eq. 28)

Substituting the measured and calculated parameters A₁, A₂, A₃ and heart rate as well as the normal Stroke Index value into Eq. 25 provides the calculated value for the flow rate or Shunt Conductance for the first right-to-left shunt, C₁ as follows:

$$C {O}_{1} = S I * H R * B S A * \frac{{A}_{1}}{{A}_{1} + {A}_{2} + {A}_{3}}$$

$$C {O}_{1} = ( 46 ml \text{/} {m}^{2} ) * ( 1.2 beats \text{/} sec ) * ( 2.06 {m}^{2} ) * \frac{45.0 mV \text{-} secs}{45.0 + 65.7 + 3100 mV \text{-} secs}$$

$$C {O}_{1} = 1.6 ml \text{/} sec$$

Likewise, substituting the measured, calculated and assumed normal Stroke Index into Eq. 26 provides the calculated value for the flow rate or Shunt Conductance for the second right-to-left shunt, C₂ as follows:

$$C {O}_{2} = S I * H R * B S A * \frac{{A}_{1}}{{A}_{1} + {A}_{2} + {A}_{3}}$$

$$C {O}_{2} = ( 46 ml \text{/} {m}^{2} ) * ( 1.2 beats \text{/} sec ) * ( 2.06 {m}^{2} ) * \frac{65.7 mV \text{-} secs}{45.0 + 65.7 + 3100 mV \text{-} secs}$$

$$C {O}_{2} = 2.3 ml \text{/} sec$$

In regards to FIG. 8, the components employed in carrying out the system and method of the invention were generally identified. In the discourse to follow the components will be described in detail following which the discussion turns to animal studies, in particular pig, and the highly desirable repeatable results of such studies. Referring to FIG. 15, the display 98 of monitor/controller and data acquisition device 82 is reproduced at a higher level of detail. In the figure, a baseline concentration dashed line is represented at 250. Above baseline concentration 250 is a filtered principal dilution curve 252. Curve 252 has been corrected for recirculation and has a peak time represented by dashed line 254. The Valsalva Maneuver is represented by a dynamic bar graph 256. Note that graph 256 is associated with measured exhalation pressure level in millimeters of mercury, the minimum value for that level being at 40 mm as represented at dotted line 258. Within graph 256 is bar 260 representing the commencement and continuation of the injection of indicator until the termination of injection as represented at dashed line 262. Note the presence of a premature inflection peak at 264. Along the display 98 are touch screen positions as well as visual cueing indicators. Region 268 is pressed by the nurse or clinician to initialize the overall procedure. Block 270 provides a cue to properly position the sensor described at 110 in FIG. 8. Block 272 cues that the system is ready for a start of the Valsalva Maneuver and corresponding block 274 indicates that the Valsalva Maneuver may be ended. A visual cue at block 276 indicates that the procedure can be repeated. In general, several tests will be run with injections from the two syringes 120 and 122. Where the clinician or nurse desires to end the procedure then touch screen 278 may be pressed. Note the presence of date, time, patients name and clinician is present along the bottom of this display.

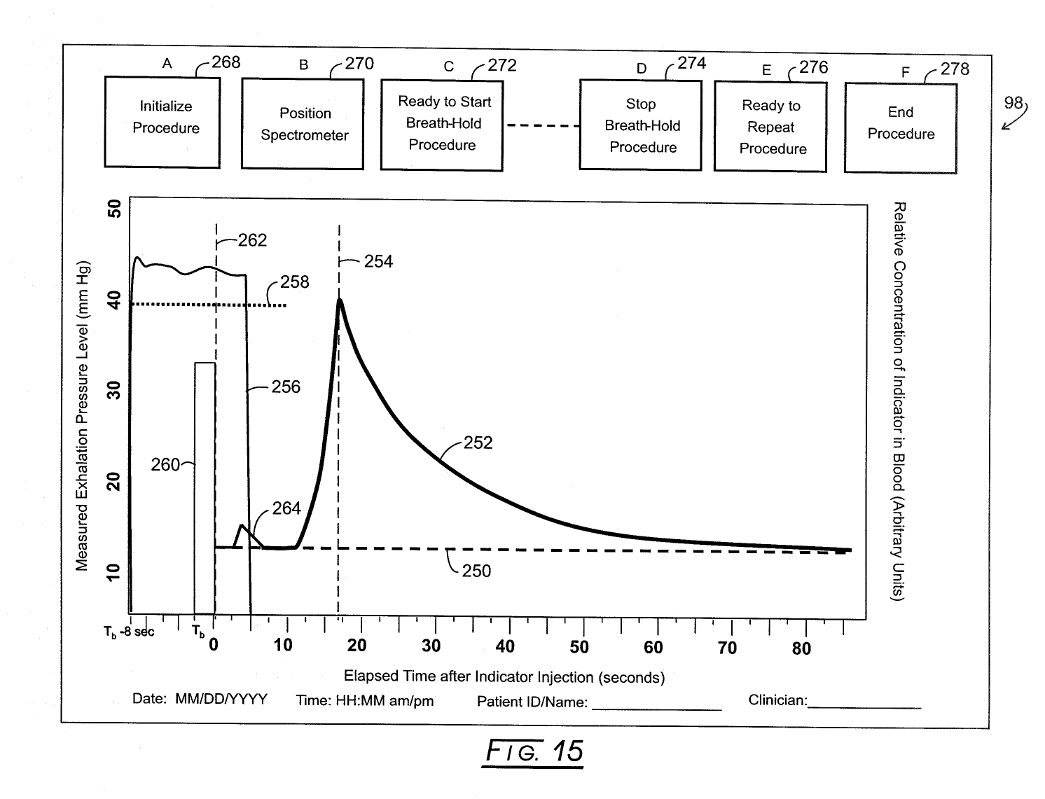

Referring to FIG. 16, an assembly view of the indicator injection assembly is presented. In the figure, first syringe 120 intended for injection of indicator reappears with that identifying numeration and similarly, the second syringe 122 intended for injection of isotonic saline flush again is identified by that same numeration. Syringes 120 and 122 are seen to be coupled to a three way stop-cock valve control represented generally again at 124 which allows the operator to select which syringe is to be used to inject indicator or isotonic saline flush.

The injected circulatory tracking reagent indicator passes through the earlier described flow sensor 126 which is controllably heated to 30° C. plus or minus 2° C. using resistance feedback control of serpentine copper adhesive material supported on a polyamide substrate, sometimes referred to as a “copper-on-Kapton”. When an injection of a liquid at room temperature occurs, the cooling effect causes an immediate decrease in the temperature of the heater component wherein a sudden decrease in temperature is used by the monitor sensing circuitry to detect the precise time at which the injection has been initiated. This allows the monitoring unit to determine if the injections of the indicator from syringe 120 and isotonic saline flush from syringe 122 are initiated within a predetermined time period after the start and before the end of the Valsalva Maneuver or test initiation. Flow sensor 126 is removably connected to a multi-lead connector 264 which in turn, is connected to earlier described cable 128 which extends to the monitor/data collector 82. In the regard to the former, note an additional connector 286, compatible with the cable connector on monitor 82.

Delivery tube 130 extends to connection with a relatively short catheter arrangement 132. Connection between delivery tube 130 and catheter arrangement 132 being made at a fitment 288. This relatively shorter catheter is employed to assure that there is no disturbance at the injected vein as a consequence of the manipulation of syringes 120 and 122 or the three way valve 124.

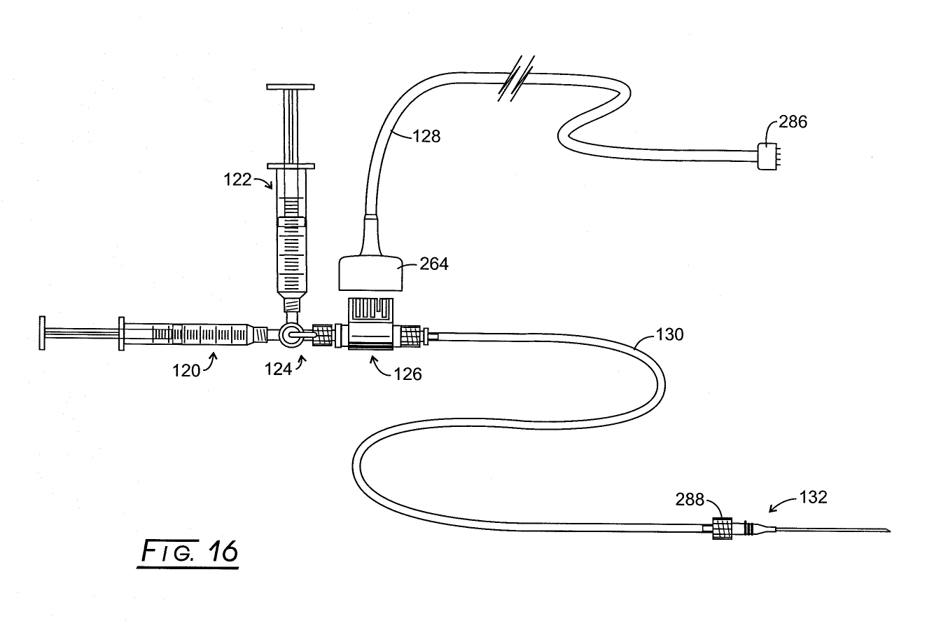

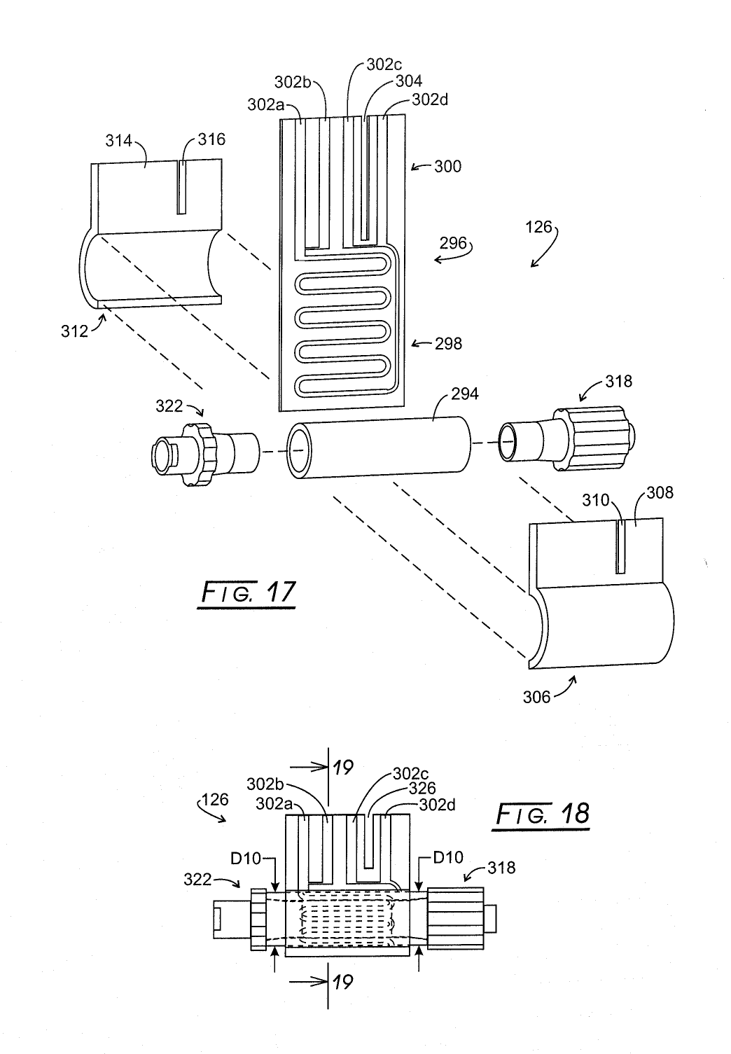

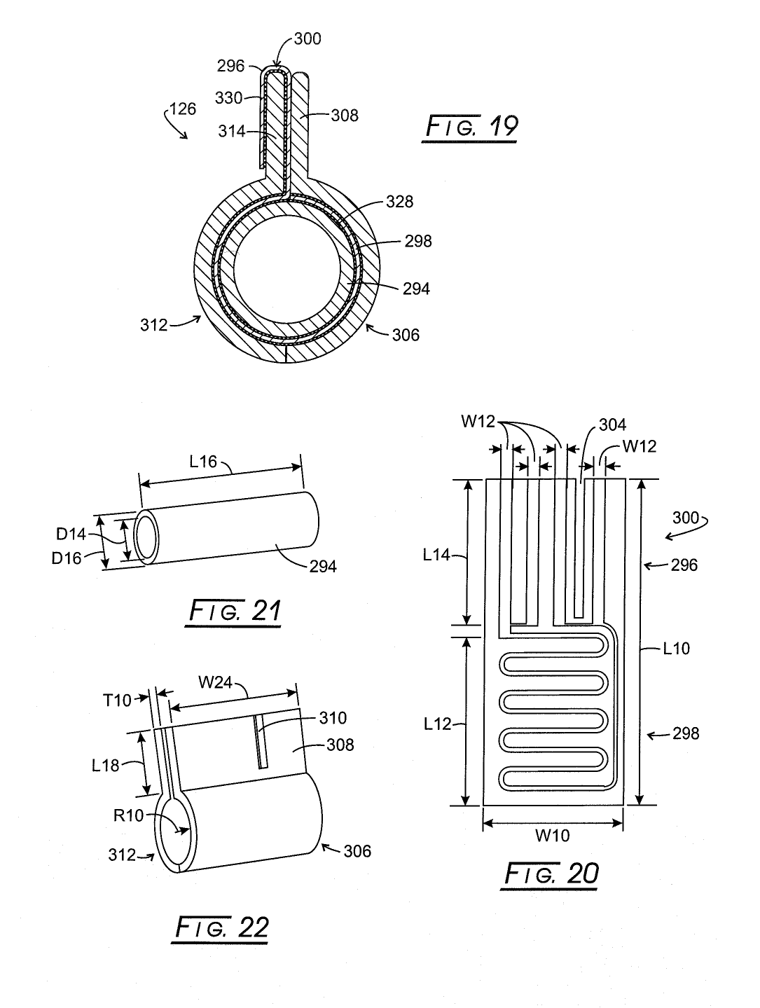

Referring to FIG. 17, an exploded view of the fluid detector 126 is set forth. The innermost component of the flow detector is a thin-walled metal tube 294, for example, formed of stainless steel type 304. Along the outside surface of tube 294 there is attached a flexible heater/lead circuit assembly represented generally at 296. Assembly 296 is comprised of a polyamide substrate referred to in the trade as “Kapton” upon which is deposited a serpentine thin copper layer represented generally at 298 and a sequence of leads represented generally at 300 and identified individually at 302a-302d. A registration slot 304 extends between leads 302c and 302b (slot 304 was intended for registration cooperation with connector 264). Of the leads within region 300, leads 302a and 302d supply current to the serpentine region 298 to derive the noted heat output. Inner leads 302b and 302c measure the voltage differential across the serpentine thin copper layer 298. The measured current flowing through said serpentine layer 298 and measured voltage differential can be used to derive resistance of serpentine layer 298. Accordingly, region 298 is physically and thermally coupled to the outer surface of tube 294 to carry this out by employing a high thermal conductance, electrically insulative transfer tape adhesive. One such transfer is tape adhesive 8805 manufactured by 3M of Minneapolis Minn. Additionally shown in the figure is an enclosure half 306 having a tongue component 308 carrying a connector registration slot 310. A corresponding enclosure half is shown in general at 312 having a tongue 314 and registration slot 316. A back end connector represented generally at 318 is inserted and adhesively connected to one end of tube 294, while an oppositely disposed connector represented generally at 322 is adhesively coupled to the opposite end of tube 294. When assembled, the upper region 300 of the circuit 296 is glued over tongue 314 to result in the arrangement shown in FIG. 18. Slots 310, 316 and 304 combine to define a registration slot 326 as seen in that figure. According to the sectional view of FIG. 19, the thermally conductive glue layer bonding circuit 296 to tube 294 is shown at 328, while the glue layer bonding the outside of circuit 296 to enclosure halves 306 and 312 as well as tongue 314 is shown at 330. For the latter coupling, cyanoacrylate adhesive may be employed.

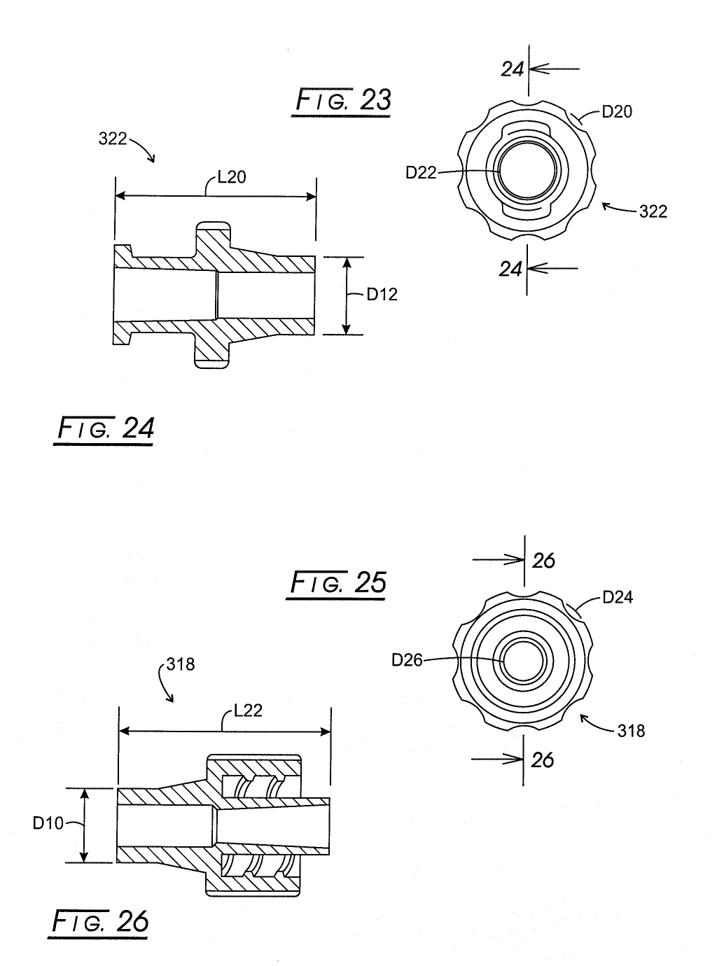

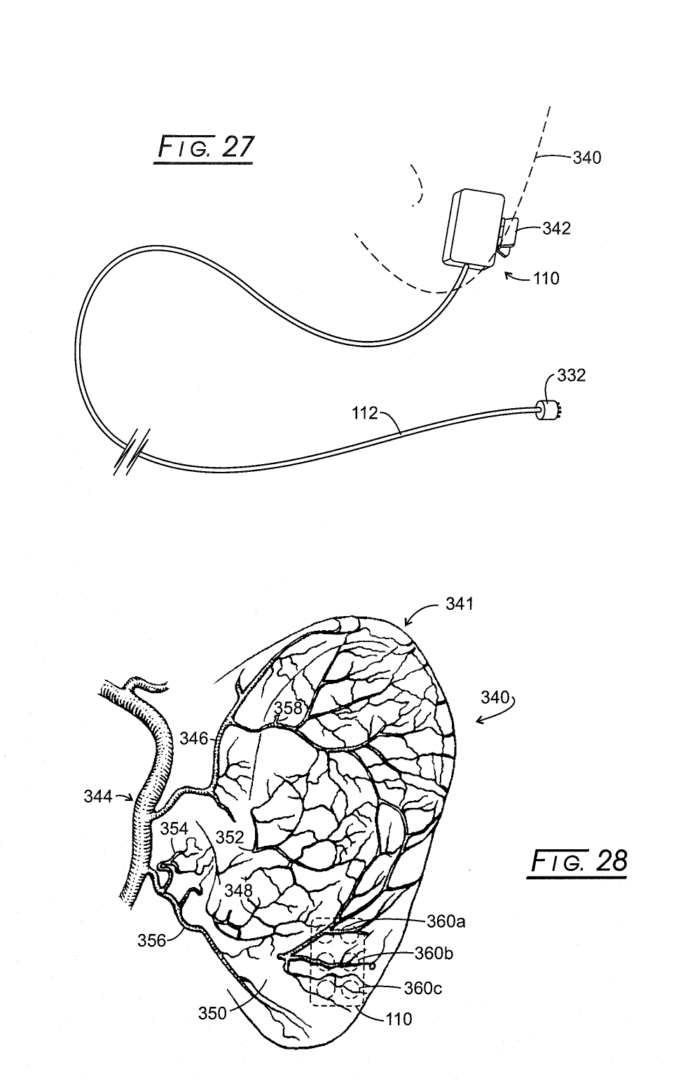

FIGS. 20-26 identify dimensions for the components of the fluid sensor. The following are preferred dimensions for that component:

L₁₀=0.8″ to 1.6″

L₁₂=0.4″ to 0.7″

L₁₄=0.4″ to 1.2

L₁₆=0.3″ to 1.0″

L₁₈=0.2″ to 0.5″

L₂₀=0.6″ to 1.4″

L₂₂=0.6″ to 1.4″

D₁₀=0.10″ to 0.25″

D₁₂=0.10″ to 0.25″

D₁₄=0.100″ to 0.250″

D₁₆=0.106″ to 0.260″

D₂₀=0.35″ to 0.60″

D₂₂=0.080″ to 0.120″

D₂₄=0.35″ to 0.60″

D₂₆=0.080″ to 0.120″

R₁₀=0.110″ to 0.265″

W₁₀=0.3″ to 1.0″

W₁₂=0.030″ to 0.060″

W₂₄=0.3″ to 1.0″

Width of heater serpentines=0.003″ to 0.010″
Thickness of Kapton in flexible heater/lead circuit=0.001″ to 0.002″
Thickness of copper in flexible heater/lead circuit=0.0003″ to 0.0010″
Thickness of high thermal conductance, electrically resistive transfer tape adhesives=0.002″ to 0.005.″

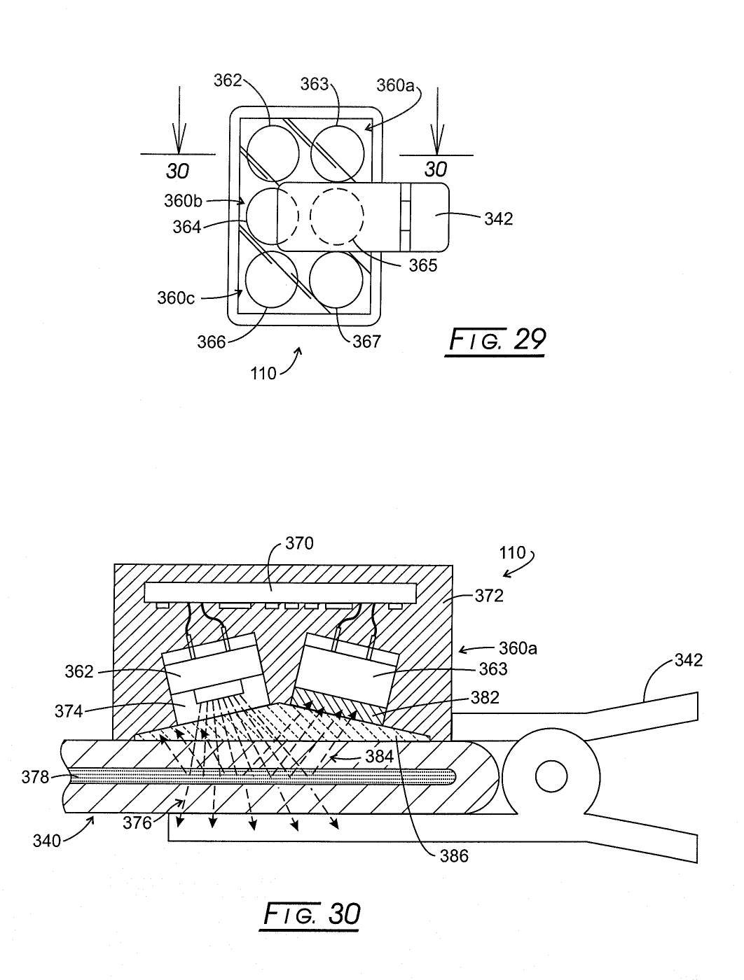

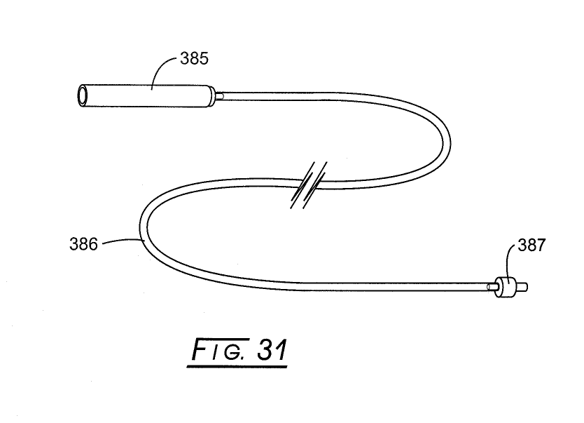

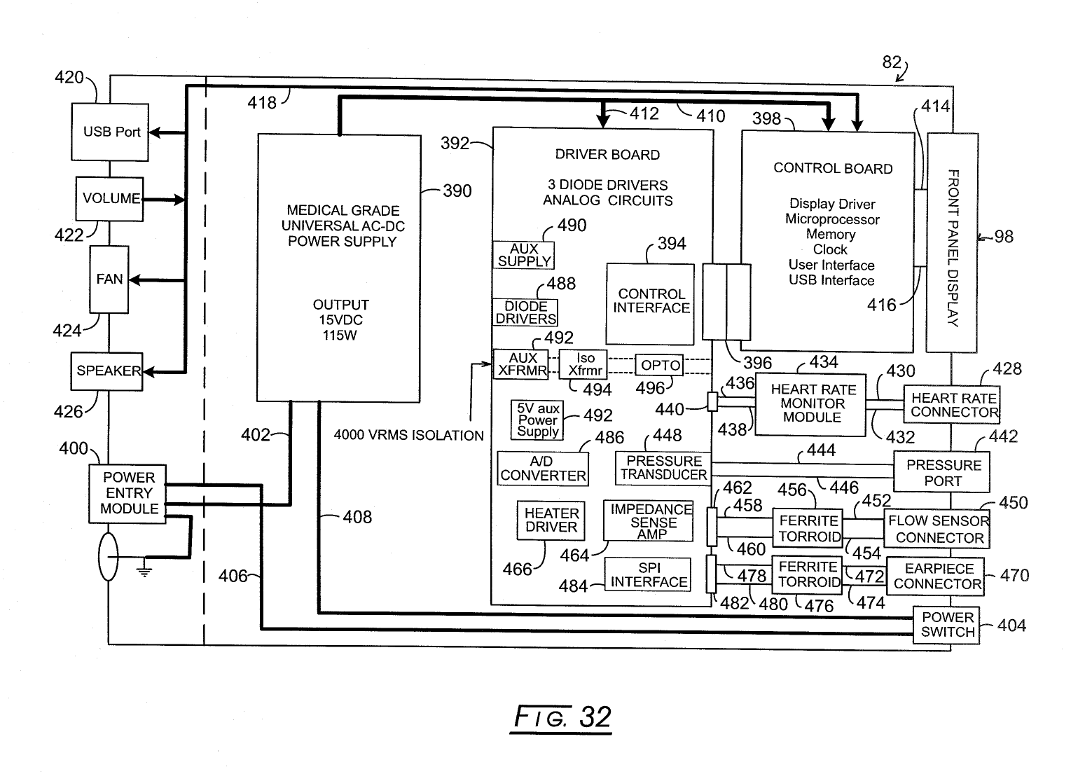

It may be recalled from FIG. 8 that the fluorescing indicator was arterially detected with a combined laser and filter photodetector arrangement attached to the pinna of the human ear. That detector was identified generally at 110 as it performed with monitor and controller 82 via cable 112. Detector 110 as well as cable 112 are reproduced at a higher scale in FIG. 27. Looking to that figure, one observes the phantom outline of a human ear represented generally at 340. To the ear 340 the sensor or detector 110 is clipped by a clipping mechanism 342. Cable 112 also is seen to be coupled with a connector 332. Ear 340 reappears in FIG. 28 in conjunction with an arterial map of the ear 341. In the latter regard, the superficial temporal artery is represented in general at 344 having an upper branch represented in general at 346, a cymbia conchae at 348, lobe at 350, middle branch 354 and lower branch 356. The triangular fossa/scapha network is represented at 358. Note in the lower region that the sensor 110 appears in phantom. In that phantom view, the sensor is seen to incorporate three paired laser/photodetectors 360a-360c. Turning to FIG. 29, of the pairs of emitters and detectors, pair 360a is seen to incorporate emitter laser 362 and photodetector/filter 363; pair 360b incorporates emitter laser 364 and photodetector/filter 365; and pair 360c incorporates emitter laser 366 and photodetector 367.

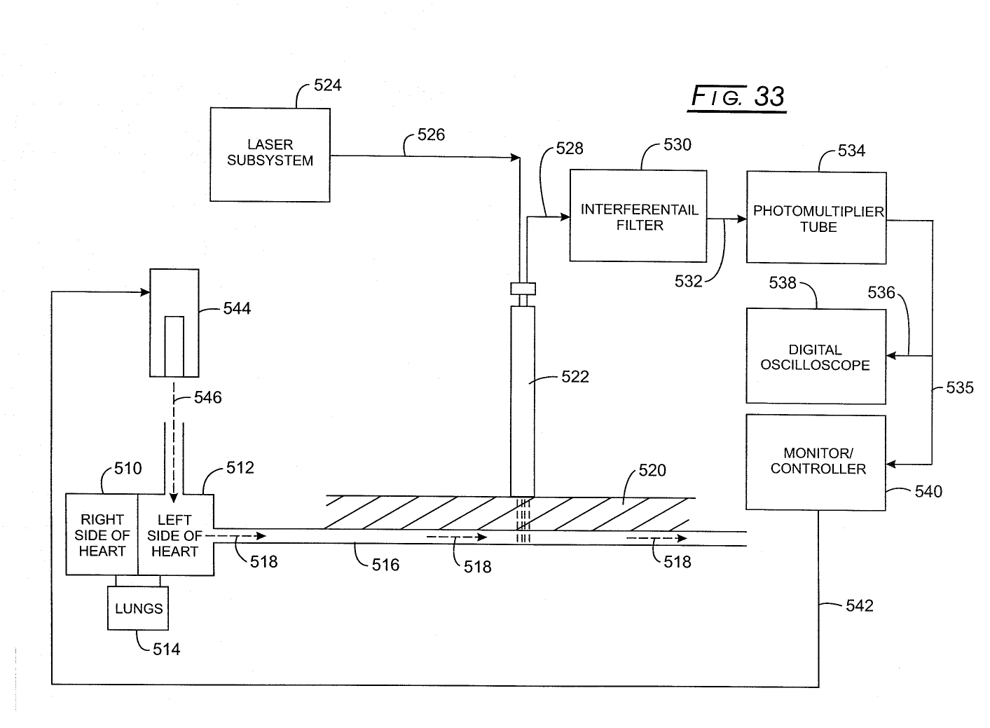

Looking to FIG. 30, laser 362 reappears being controllably energized from the components of a printed circuit board 370 within support housing 372. The laser is within a circular cavity 374 and is illustrated as being energized to create laser light represented by arrow array 376 penetrating the flesh and arteries at ear 340. One or more arteries are represented at 378. Adjacent to laser 362 is photodetector 363 and forwardly disposed interferential filter 382 which receives and passes fluorescing photons as represented at arrow array 384. Printed circuit board 370 may incorporate such features as amplification stages and analog-to-digital conversion functions. Devices 362 and 363 perform in conjunction with a triangular lens 386. Practitioners generally will apply an optical coupling agent, such as an optical gel between the ear and lens 386.

Referring now to the exemplary embodiment shown in FIG. 28, three laser/photodetector pairs 360a-360c are positioned at the surface of the skin overlying blood vessels within the ear. Said blood vessels are not visible at the skin surface due to the intervening thickness of tissue between the skin surface and the blood vessels within the ear. In the preferred embodiment shown in FIGS. 28, 29 and 30, the use of multiple laser/photodetector pairs allows laser light 376 and resulting fluorescence photons 384 from each pair to be sequentially emitted and received, respectively thereby obtaining a measurement of indicator concentration at three distinct locations as seen in FIG. 28. By way of example, a first laser/photodetector pair 360a comprising laser 362 and photodetector/filter 363 as seen in FIGS. 28, 29 and 30 is sequentially interrogated by energizing laser 362 to generate laser light 376 for a fixed time period ranging from 1 to 20 milliseconds while measuring the emitted fluorescence photons 384 with photodetector/filter 363 during the same fixed time period ranging from 1 to 20 milliseconds. Immediately following this first time period, a second laser/photodetector pair 360b comprising laser 364 and photodetector/filter 365 as seen in FIGS. 28, 29 and 30 is sequentially interrogated by energizing laser 364 to generate laser light for a fixed time period ranging from 1 to 20 milliseconds while measuring the emitted fluorescence photons with photodetector/filter 365 during the same fixed time period ranging from 1 to 20 milliseconds. Immediately following this second time period, a third laser/photodetector pair 360c comprising laser 366 and photodetector/filter 367 as seen in FIGS. 28, 29 and 30 is sequentially interrogated by energizing laser 366 to generate laser light for a fixed time period ranging from 1 to 20 milliseconds while measuring the emitted fluorescence photons with photodetector/filter 367 during the same fixed time period ranging from 1 to 20 milliseconds. This sequential energizing of the laser and measuring the emitted fluorescence photons with the photodetector/filter in each pair of this three-pair array is repeated continuously throughout the 30 to 60 second period of measuring the concentration of the fluorescing indicator within the blood vessels within the ear in the region of the laser/photodetector pairs 360a-360c. The laser/photodetector pair which yields the highest measured peak fluorescence photon level during the 30 to 60 second measurement period will be selected to display the relative indicator concentration level as a function of time as seen in FIG. 15. According to this method, the present invention allows the selection of that laser/photodetector pair which is in the closest proximity to the largest blood vessel or blood vessel array even though the blood vessels can not be visually observed during the placement of the sensor or detector 110 at a fixed position at the skin surface of the patient (e.g., the pinna of the ear). As a result, the emitted photon signal level obtained from the fluorescing indicator within the blood stream can be maximized thereby increasing the sensitivity the method to detect smaller right-to-left shunts as well as increasing the accuracy of the quantification of the Shunt Conductance.

This use of an array of two or more sensors or detectors can also be applied to the detection of the concentration of indicators in the blood stream other than fluorescing indicators. For example, this same arrangement of two or more sensors or detectors can be applied to relative indicator concentration measurement methods involving (a) infrared spectroscopy, (b) radiation detection for the case of radio-labeled indicators, (c) radiographic detection of radio-opaque indicators, (d) magnetic resonance based detection involving magnetic resonance imaging contrast agents and/or (e) ultrasound detection methods involving ultrasound contrast agents.

In addition and still referring to FIG. 28, the blood vessels in the region of the placement of the detector or sensor 110 may be dilated to increase blood flow and thereby to increase signal level of detectable indicator within the blood stream. This process is widely known as vasodilation and can be accomplished by one or a combination of several methods including (a) pre-heating the tissue at the location of sensor or detector 110 to about 40 to 42 C for a period of several minutes up to 10 minutes prior to the start of the injection of the indicator and/or (b) application of pharmacologic agents such as capsaicin which induce the dilation of blood vessels without the application of an external source of heat.

The sensor is embodied in a sensor apparatus comprising (a) an emitter-detector pair for monitoring the fluorescence of a fluorescing circulatory tracking reagent; b) an emitter providing a light source emitting a first wavelength for the transcutaneous excitation of an indicator within the bloodstream; and (c) a detector for measuring the intensity of the light emitted at a second wavelength from an indicator within the blood stream. The apparatus typically will be embodied with a plurality of emitter and detector pairs as disposed in a sensor array. The plurality of emitter and detector pairs allows the sensors to be sequentially queried in order to determine the emitter and detector pair providing a preferred sensor from the sensor array. Particularly, it is expected that one emitter/detector pair will be in a preferred proximity to the arterial blood flow of the sensor location. The sensor is a normally utilized as a transcutaneous sensor and the preferred sensor is determined by identifying the sensor in closest relationship a subcutaneous blood vessel in order to maximize the sensitivity of said sensor. The preferred sensor may be determined by one or more of signal to noise ratio, absolute signal level, and minimum background signal. As shown, the sensor apparatus is preferably utilized with an optical coupling agent at the skin/instrument interface. Moreover, the sensor apparatus can be queried for the reflectance of the emitter signal from the skin surface, and thereby used to determine the background radiation level and the absence of reflected signal from the skin triggers a no signal fault indication.

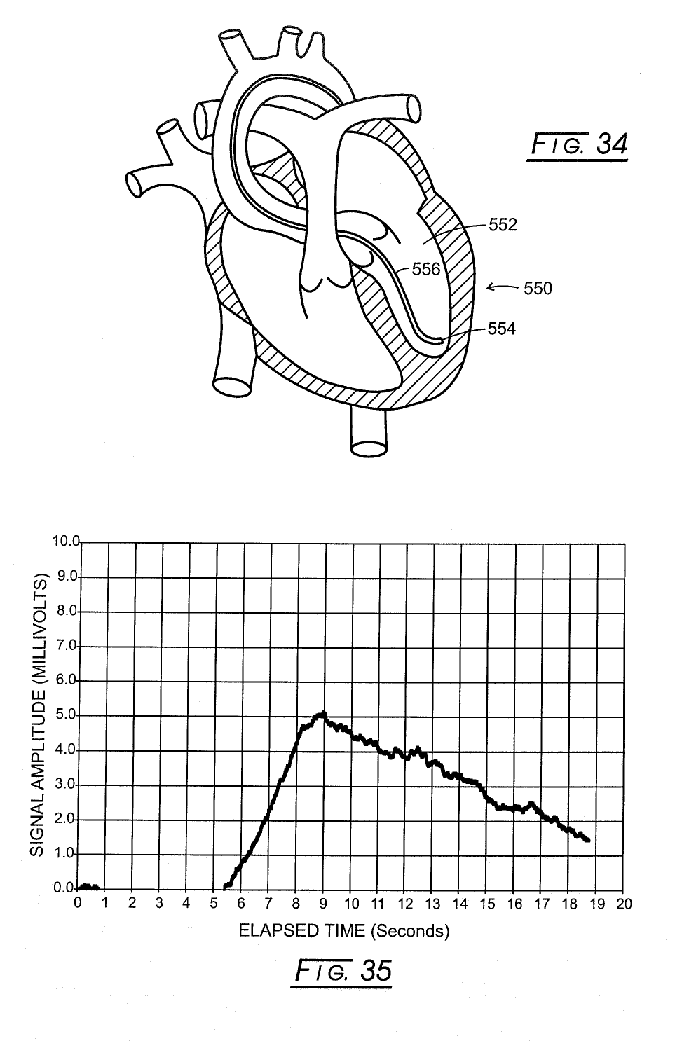

Referring to FIG. 31, an assembly view of the Valsalva Maneuver pressure sensing unit is seen which comprises mouthpiece 385. Mouthpiece 385 is formed of rigid polypropylene tubing having an outside diameter of 0.375 inch, inside diameter of 0.250 inch and overall length of 1.50 inch and extension tubing 386. Extension tubing 386 is vinyl tubing having an outside diameter of 0.250 inch and inside diameter of 0.125 inch with an overall length of 72 inches. The mouthpiece 385 is attached to the tubing 386 with a fitment (not shown) with barbed mechanical attachment and/or gas-tight adhesive attachment. The proximal end 387 of extension tubing 386 is terminated with a quick-disconnect fitment 387 such as Quick-Disconnect Insert Fitting with Hose Barb fitting for securing to extension tubing supplied by Cole Parmer, Chicago, Ill., catalog No. K-06360-42. This quick-disconnect fitment 387 attaches to mating quick disconnect port at monitor 82 which, in turn, is connected to a pressure transducer within the monitor to continuously measure exhalation pressure exerted by a patient into mouthpiece 385 during a Valsalva Maneuver. Recall that the display as described in connection with FIG. 15 includes a dynamic bar graph which shows the appropriate level for 40 mmHg during the 15-20 second period of the described Valsalva Maneuver. An audible warning is given should minimum pressure be dropped during such maneuver.

Referring to FIG. 32, a block schematic representation of the monitor/controller/data acquisition system is presented again identified by the general numeration 82. In general, the apparatus 82 includes a medical grade universal AC-DC power supply 390 having a 15 v D.C. output at 115 watts. Adjacent the power supply 390 is a driver board 392. Driver board 392 is operatively associated through a control interface 394 and coupling 396 with a control board 398.

Power supply 390 receives input power via entry module 400, cable 402; an input power switch 404 is represented at cable 406. The opposite input from power switch 404 is at cable 408 which is directed to power supply 390. The output from power supply 390 is present at cables 410 and 412 extending respectively to control board 398 and driver board 392. Control board 328 is seen to incorporate a display driver; microprocessor; memory; clock; user interface; and USB interface. It is associated with the front panel display 98 as represented at lines 414 and 416. The control board 398 also is operationally associated via bi-directional bus 418 with a USB port 420; a volume control 422; a fan 424 and a speaker 426.

The heart rate monitor shown in FIG. 8 at 114 is coupled with a heart rate connector shown in the instant figure at 428. Connector 428 is coupled as represented at lines 430 and 432 with a heart rate monitor module 434 which in turn is coupled with the driver board as represented at lines 436 and 438 as well as connector 440.

Tubing for carrying out the Valsalva Maneuver has been described in FIG. 8 at 106 and 108. As tubing couples with a pressure port 442 which is operationally associated as represented by lines 444 and 446 with a manometer or pressure transducer 448 located upon driver board 392. Next, leads from the flow sensor 126 extend to a flow sensor connector represented at 450. Flow sensor connector 450 extends as represented at lines 452 and 454 through a ferrite torroid 456, thence via lines 458 and 460 and connector 462 to an impedance sense amplification stage 464 and a heater driver 466. It may be recalled that the temperature of the heater in the fluid sensor is monitored in a resistance manner owing to the high temperature coefficient of resistance of the copper serpentine heater 298.

Cable 112 from earpiece 110 couples with an earpiece connector as represented at block 470. Connector 470 is directed as represented at lines 472 and 474 through a ferrite torroid 476 and lines 478 and 480 to connector 482 which is associated with signal processing interface 484. It may be recalled that the analog signals from the photodetector are filtered and subjected to fast Fourier transform activity that involves A/D conversion as represented at block 486. Additionally, the laser diodes are driven by diode drivers represented at block 488 which perform in conjunction with such details as an auxiliary supply 490 and auxiliary transformer 492.

Also shown at the driver board 392 is an auxiliary transformer function 492; an isolation transformer function 494 and an opto coupling function 496.

It should be borne in mind that the earpiece paired lasers and photodetectors are serially polled to find a most significant signal which assures that an arterial association of a given pair will be an optimum one.

### Example 3

### System and Method for Testing Circulatory Tracking Indicators and Detectors

The present disclosure is also embodied in a method for testing systems for monitoring cardiac output, circulatory behavior of blood fluids, and blood circulation, including circulation within peripheral tissues of a human body and organs, such as the heart, brain or liver. In particular, a method for utilizing an experimental animal body for determining the efficacy of circulatory tracking systems by emplacing an injection catheter into the circulatory system or a chamber of the heart in a test animal with a functioning circulatory system and heart. Once the injection catheter is emplaced, a number of variables in a circulatory tracking system to be tested may be altered. For instance, a series of circulatory tracking reagents being tested with the method may be injected in the circulatory system of the test animal, and detector systems compatible with the circulatory tracking reagent can be activated at given locations on the body of the test animal. Then, the monitoring of the efficacy of a combination of given detectors detector locations and circulatory tracking reagents to detect the presence of given circulatory tracking agents at a particular location on the test animal body allows the optimization of a given circulatory tracking system.

In a specific example, to determine the efficacy of indicator dyes, a bolus of circulatory tracking reagent is injected into the circulatory system or chamber of the heart, and the detector is emplaced on the body of the test animal such that the transit of the indicator dye may be traced.

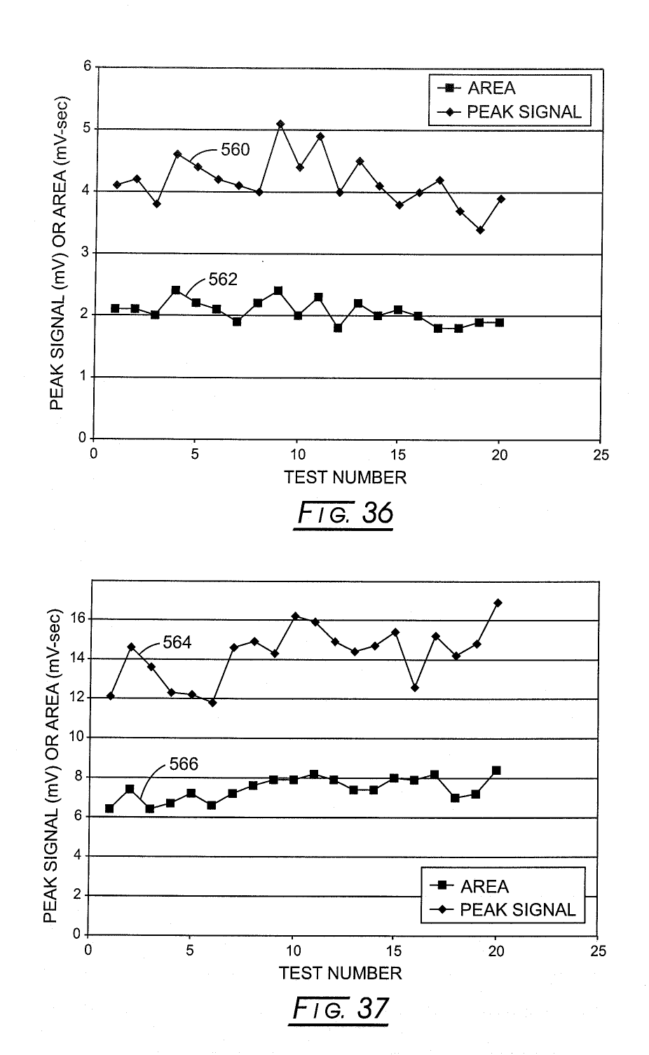

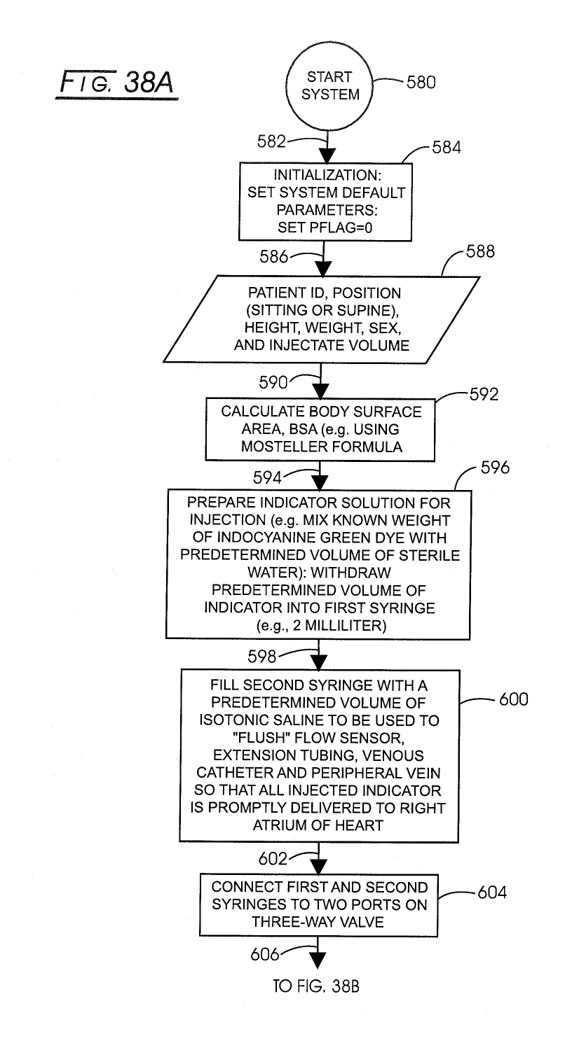

The above disclosed system, apparatus and method utilized an animal model in order to test the efficacy of the method and apparatus. The carrying out of effective animal testing of the above described system and method was not a trivial undertaking. Implementing a right-to-left shunt within the cardiopulmonary system was not practical as well as somehow emulating a Valsalva Maneuver would not be effective on an animal (e.g., pig) maintained on or under general anesthesia. Looking to FIG. 33, a schematic sketch of the technique developed is presented. In the figure, the right and left sides of an animal's heart are represented respectively at blocks 510 and 512. Blocks 510 and 512 are represented as being associated with the lungs of the animal represented at block 514. Arterial vascularity as extending from the left side of the heart is represented by conduit 516 and the movement therethrough of injected indicator and blood is represented at arrow 518. Tissue overlying blood vessel conduit 516 is represented at 520 and a combination laser exciter and fluorescing photodetection component is represented at 522 with end view of component 522 shown in FIG. 3. Laser excitation into component 522 is represented at block 524 and arrow 526, while the fluorescence output is shown directed from component 522 at arrow 528, whereupon it is filtered at interferential filter 530, the filtered output of which is shown at arrow 532 as being directed to photodetection as represented at block 534. The photodetected output is alternately directed as represented at arrow 536 to a digital oscilloscope represented at block 538 or as represented at arrow 535 to a monitor controller 540. As represented at arrow 542, symbol 544 and arrow 546, a dosage indicator was controllably injected into the left ventricle of the animal heart with a concentration and dosage emulating a right-to-left shunt. To make this injection, a pigtail catheter was utilized. Looking momentarily to FIG. 34, the heart of a pig is schematically illustrated and identified generally at 550. The left ventricle is shown at 552 and the outlet or tip 554 of catheter 556 is shown within left ventricle 552.

The general protocol for the in vivo animal experiments was as follows. Four female swine (pig), weighing 80-100 pounds were selected. Preoperative sedation was either telazol or xylazine, 1 g IM. Following sedation the animal was anesthetized via inhalation anesthesia (isoflurane 0.7-3%, initially) in oxygen via nose cone. Flurane was titrated to maintain a surgical plane of anesthesia (abolition of the lateral canthal reflex and lack of hypertension or tachycardia) throughout the procedure. A tracheostomy was performed and the animal was ventilated. Fluid filled catheters for pressure tracings and blood gases were placed by cutdown in the carotid artery and jugular vein to aid in maintaining homeostasis and monitoring adequacy of anesthesia and volume administration. Intravenous anesthesia such as fentanyl and/or pentothal was used as necessary.

Percutaneous access was established in a femoral artery using a 5 French, 90 cm pig-tail catheter (Cook Royal Flush Plus) fed retrograde into the left ventricle of the heart. Confirmation of catheter placement in the left ventricle was performed with fluoroscopic method and monitoring of pressure waveforms. Once left ventricle placement was confirmed, a transcutaneous fluoroscopic sensor unit was placed in contact with the skin surface at the ear. A syringe pump was used to deliver a range of doses of ICG ranging from approximately 0.016 ml to 2 ml of volume per injection at concentrations ranging from 0.4 to 1.6 mg/ml to allow for measurement of peak fluorescence signal as a function of injected ICG dose. This range of ICG dose levels was selected to simulate the range of magnitudes of right-to-left shunt “leakage rates” or fluid conductances.

A number of parameters were maintained at constant levels during the in vivo pig experiments. As a fluorescence detection system was utilized, excitation laser power level was at 100 milliwatts. The injection duration was approximately 1.1 seconds, and the ICG dye concentration: 400 micrograms/ml (or μg/ml), while the fluorescence probe was positioned proximal to either a blood vessel in the left ear or blood vessel in the right ear. An optical coupling agent, Aquasonic Gel, was utilized at the probe/skin interface.

Initially measurements were taken following introduction of an ICG bolus of peak fluorescence signal amplitude compared with dose levels in order to determine proper positioning of the sensor probe. Peak signal amplitude was measured with a 100 ®g dose delivered with a with 10 ml volume via syringe. Once it was determined that an operable sensor site was located, 20 repetitions at a 25 ®g dose (0.063 ml injection volume) at interval of about 1 minute between injections. The injection and monitoring was repeated utilizing a 12.5 μg dose (0.0315 ml injection volume), and utilizing a 6.4 ®g dose (0.0160 ml injection volume).

Next the effect of injection volume was investigated, by determining peak fluorescence signal amplitude with a fixed dose, and variable bolus volumes. Two different doses were repeatedly delivered in either a 1 ml volume or a 10 ml volume.

Following the animal testing protocol, euthanasia was performed with 40 mEq of potassium chloride via a central venous line at the conclusion of the testing.

Looking momentarily to FIG. 35, an indicator dye dilution curve carried out with respect to a 6.4 microgram dose; an injection period of 1.1 seconds and an ICG concentration of 400 micrograms per millimeter with an injection volume of 0.016 milliliters is set forth. In particular, the area under such curves in mV seconds and consistent doses of ICG were recorded. Laser power was held at 100 milliwatts.

A parametric analysis of the test results was used to determine the ICG dose required for the high sensitivity detection of simulated right-to-left shunts of various sizes using the transcutaneous fluoroscopic sensor unit. Looking to Table 1, test data or test numbers 091 to 130 are set forth. Set forth in the table, for example, is the area under the shunt curve and millivolt-seconds as well as the ICG dose. The area under the dye dilution curve as well as peak signals were plotted for tests 091-110 and 111-130. The peak signals of test numbers 091-110 are plotted and in FIG. 36 at curve 560, while the corresponding area under the dye dilution curve, for example, as seen at FIG. 36 is provided at curve 562.

TABLE 1
Sample data for in vivo animal testing.

| | | | | | | | | Mean | |
|---|---|---|---|---|---|---|---|---|---|
| | | | ICG | | Volume | Inj. | | BP | Heart |
| | Peak | AUC | Dose | Target | Injected | Rate | Inj. Dur. | mm | Rate |
| Test | [mV] | [mVs] | [mg] | Location | [ml] | ml/min | (s) | Hg | (BPM) |
| 091 | 4.1 | 2.1 | 0.0064 | REV distal | 0.016 | 0.87 | 1.1034 | 62 | 93 |
| 092 | 4.2 | 2.1 | 0.0064 | REV distal | 0.016 | 0.87 | 1.1034 | 62 | 93 |
| 093 | 3.8 | 2.0 | 0.0064 | REV distal | 0.016 | 0.87 | 1.1034 | 62 | 93 |
| 094 | 4.6 | 2.4 | 0.0064 | REV distal | 0.016 | 0.87 | 1.1034 | 62 | 93 |
| 095 | 4.4 | 2.2 | 0.0064 | REV distal | 0.016 | 0.87 | 1.1034 | 62 | 93 |
| 096 | 4.2 | 2.1 | 0.0064 | REV distal | 0.016 | 0.87 | 1.1034 | 62 | 93 |
| 097 | 4.1 | 1.9 | 0.0064 | REV distal | 0.016 | 0.87 | 1.1034 | 61 | 94 |
| 098 | 4.0 | 2.2 | 0.0064 | REV distal | 0.016 | 0.87 | 1.1034 | 61 | 94 |
| 099 | 5.1 | 2.4 | 0.0064 | REV distal | 0.016 | 0.87 | 1.1034 | 61 | 94 |
| 100 | 4.4 | 2.0 | 0.0064 | REV distal | 0.016 | 0.87 | 1.1034 | 61 | 94 |
| 101 | 4.9 | 2.3 | 0.0064 | REV distal | 0.016 | 0.87 | 1.1034 | 61 | 94 |
| 102 | 4.0 | 1.8 | 0.0064 | REV distal | 0.016 | 0.87 | 1.1034 | 61 | 94 |
| 103 | 4.5 | 2.2 | 0.0064 | REV distal | 0.016 | 0.87 | 1.1034 | 61 | 94 |
| 104 | 4.1 | 2.0 | 0.0064 | REV distal | 0.016 | 0.87 | 1.1034 | 61 | 94 |
| 105 | 3.8 | 2.1 | 0.0064 | REV prox | 0.016 | 0.87 | 1.1034 | 61 | 94 |
| 106 | 4.0 | 2.0 | 0.0064 | REV prox | 0.016 | 0.87 | 1.1034 | 61 | 94 |
| 107 | 4.2 | 1.8 | 0.0064 | REV prox | 0.016 | 0.87 | 1.1034 | 61 | 94 |
| 108 | 3.7 | 1.8 | 0.0064 | REV prox | 0.016 | 0.87 | 1.1034 | 60 | 95 |
| 109 | 3.4 | 1.9 | 0.0064 | REV prox | 0.016 | 0.87 | 1.1034 | 60 | 95 |
| 110 | 3.9 | 1.9 | 0.0064 | REV prox | 0.016 | 0.87 | 1.1034 | 60 | 95 |
| 111 | 12.1 | 6.4 | 0.0126 | REV prox | 0.0315 | 1.72 | 1.0988 | 60 | 95 |
| 112 | 14.6 | 7.4 | 0.0126 | REV prox | 0.0315 | 1.72 | 1.0988 | 60 | 95 |
| 113 | 13.6 | 6.4 | 0.0126 | REV prox | 0.0315 | 1.72 | 1.0988 | 60 | 95 |
| 114 | 12.3 | 6.7 | 0.0126 | REV prox | 0.0315 | 1.72 | 1.0988 | 60 | 95 |
| 115 | 12.2 | 7.2 | 0.0126 | REV prox | 0.0315 | 1.72 | 1.0988 | 60 | 95 |
| 116 | 11.8 | 6.6 | 0.0126 | REV prox | 0.0315 | 1.72 | 1.0988 | 62 | 172 |
| 117 | 14.6 | 7.2 | 0.0126 | REV prox | 0.0315 | 1.72 | 1.0988 | 62 | 172 |
| 118 | 14.9 | 7.6 | 0.0126 | REV prox | 0.0315 | 1.72 | 1.0988 | 62 | 172 |
| 119 | 14.3 | 7.9 | 0.0126 | REV prox | 0.0315 | 1.72 | 1.0988 | 62 | 188 |
| 120 | 16.2 | 7.9 | 0.0126 | REV prox | 0.0315 | 1.72 | 1.0988 | 62 | 188 |
| 121 | 15.9 | 8.2 | 0.0126 | REV prox | 0.0315 | 1.72 | 1.0988 | 62 | 188 |
| 122 | 14.9 | 7.9 | 0.0126 | REV prox | 0.0315 | 1.72 | 1.0988 | 60 | 100 |
| 123 | 14.4 | 7.4 | 0.0126 | REV prox | 0.0315 | 1.72 | 1.0988 | 60 | 100 |
| 124 | 14.7 | 7.4 | 0.0126 | REV prox | 0.0315 | 1.72 | 1.0988 | 60 | 100 |
| 125 | 15.4 | 8.0 | 0.0126 | REV prox | 0.0315 | 1.72 | 1.0988 | 60 | 100 |
| 126 | 12.6 | 7.9 | 0.0126 | REV prox | 0.0315 | 1.72 | 1.0988 | 60 | 100 |
| 127 | 15.2 | 8.2 | 0.0126 | REV prox | 0.0315 | 1.72 | 1.0988 | 60 | 100 |
| 128 | 14.2 | 7.0 | 0.0126 | REV prox | 0.0315 | 1.72 | 1.0988 | 60 | 100 |
| 129 | 14.8 | 7.2 | 0.0126 | REV prox | 0.0315 | 1.72 | 1.0988 | 61 | 102 |
| 130 | 16.9 | 8.4 | 0.0126 | REV prox | 0.0315 | 1.72 | 1.0988 | 61 | 102 |

In Table 1, the column headings are abbreviated as follows: “Test” represents the individual test number; “Peak [mV]” is the measured peak amplitude in millivolts; “AUC [mVs]” represents the integrated area under the curve in millivolt-seconds; “ICG Dose [mg]” is the indocyanine green dose in milligrams; “Target Location” represents the location the detector was placed, either right ear at a distal or proximal location; “Volume Injected [ml]” represents the volume of indicator injected in ml of 400 μg/ml ICG; “Inj. Rate ml/min” represents the measured injection rate of the indicator solution in milliliters/minute; “Inj. Dur. (s)” represents the measured duration of the injection in seconds; “Mean BP mm Hg” represents the mean diastolic blood pressure in millimeters of mercury; and “Heart Rate (BPM)” represents the measured heart rate in beats per minute.

Looking to FIG. 37, the same form of data is plotted for test 111-130. In this regard, curve 564 plots peak signal for those tests and curve 566 plots the area under the dye dilution curve for those tests. The test numbers 091-110 and 111-130 as set forth at Table 1 are replaced with the numbers 1-20 in FIG. 36 and FIG. 37, respectively.

As the data in Table 1 and the associated figures demonstrate, various dye combinations may be injected directly into the porcine heart, and the ability to detect that indicator may be tested using a variety of detector combinations. Although fluorescent indicators and in particular ICG in combination with a fluorescence excitation and detection systems are described in detail herein, the invention is embodied in a method of testing various additional combinations utilizing a animal model as described.

For example, additional fluorescent circulatory tracking reagents may be tested utilizing similar sensors to those shown in FIGS. 3-5 and 29-30, with the appropriate emitters and detectors. Moreover, the efficacy of various locations of placement of the sensor apparatus may be readily tested using the animal model and a controlled introduction of circulatory tracking reagent.

Additional non-fluorescent circulatory tracking reagents may also be tested utilizing the described animal model. Spectrophotometric and or densitometric indicators may similarly be tested simply by altering the emitter and detector frequencies to utilize the known properties of such agents in blood. Radioactive isotopes, for instance, are amenable for use as a circulatory tracking reagent, and effective use would rely on alteration of the sensor apparatus in order to detect such agents. A variety of radioactive detectors are known to those skilled in the art of radiology, as are rapidly metabolizable radioactive reagents that could be utilized with the disclosed system, method and apparatus. Previously, the ability to utilize the wide variety of potential circulatory tracking reagents has been severely limited by the inability to reproducibly invoke the opening of shunts, and even to perform such testing in humans at all.

A number of different animals in addition to pig may readily be utilized using the animal model demonstrated herein. Any mammal potentially could be utilized, while mammals with a heart of the approximate size of a human heart are preferred, and may include primates, i.e. rhesus monkey, chimpanzee, canines, and felines. Large rodents are predicted to be amenable to the technique, yet in the typical laboratory rodent, the small size of veins, heart and circulating blood volume are believed to limit the ease of use of the technique for testing circulatory tracking reagents and compatible sensors.

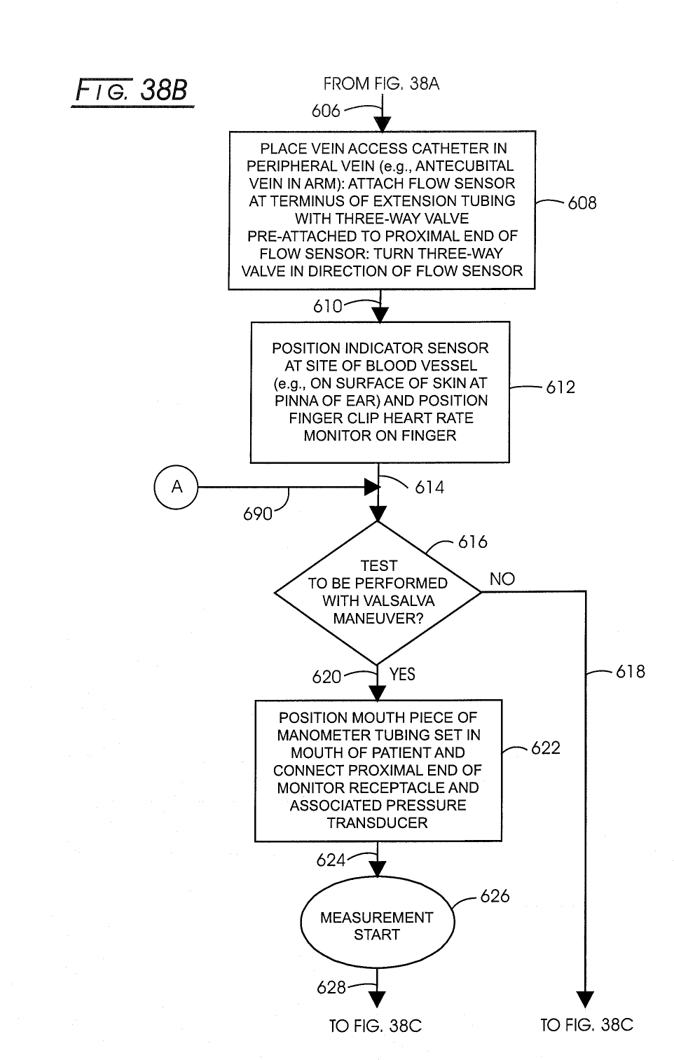

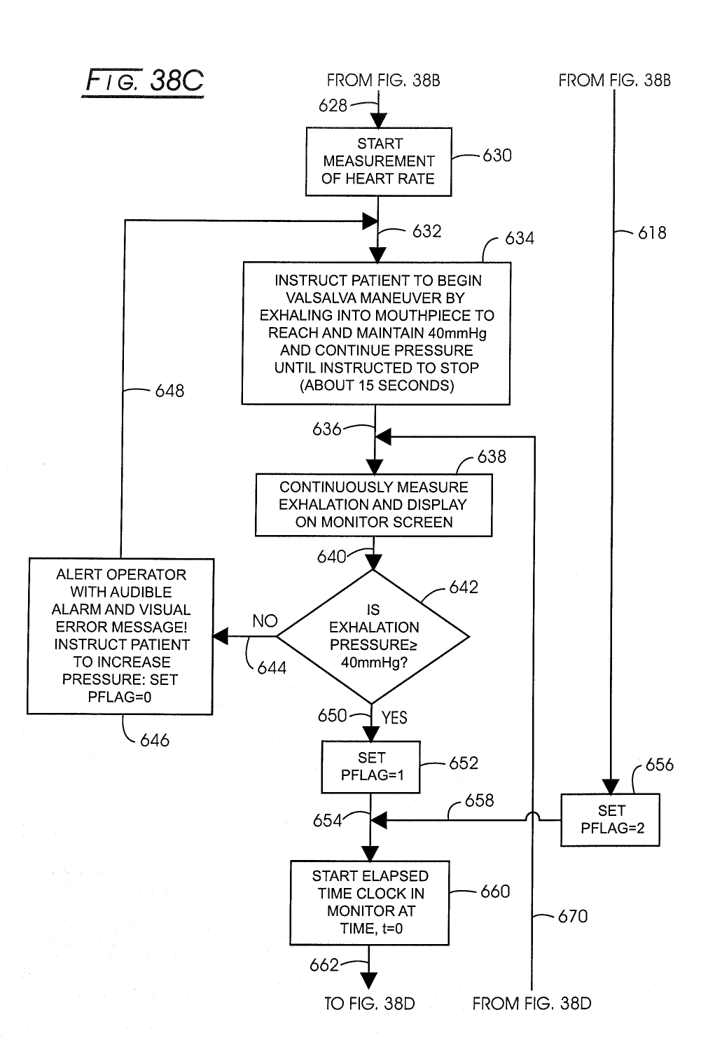

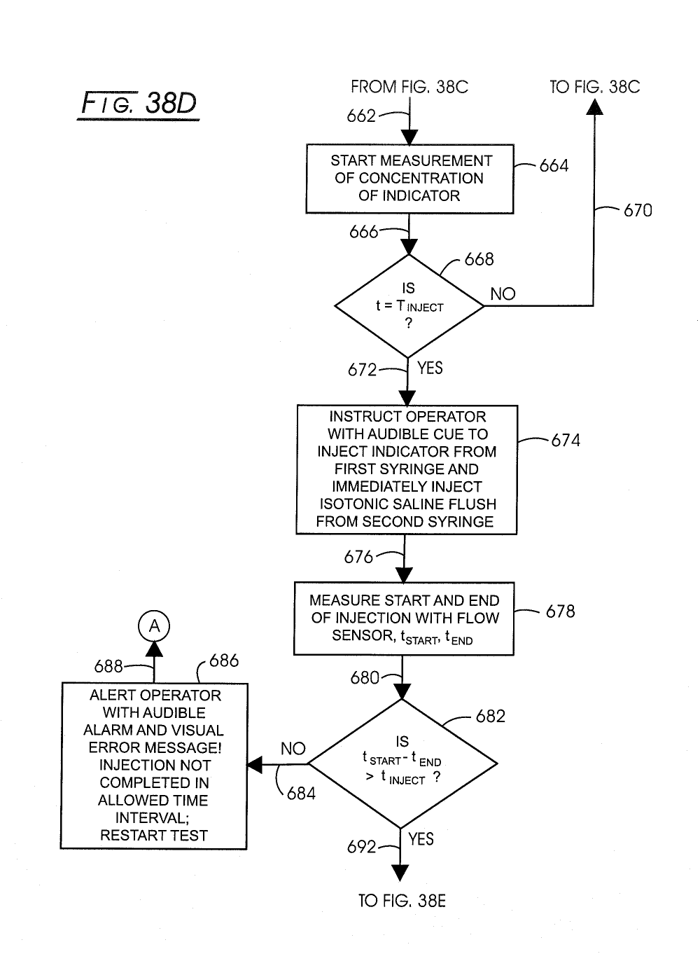

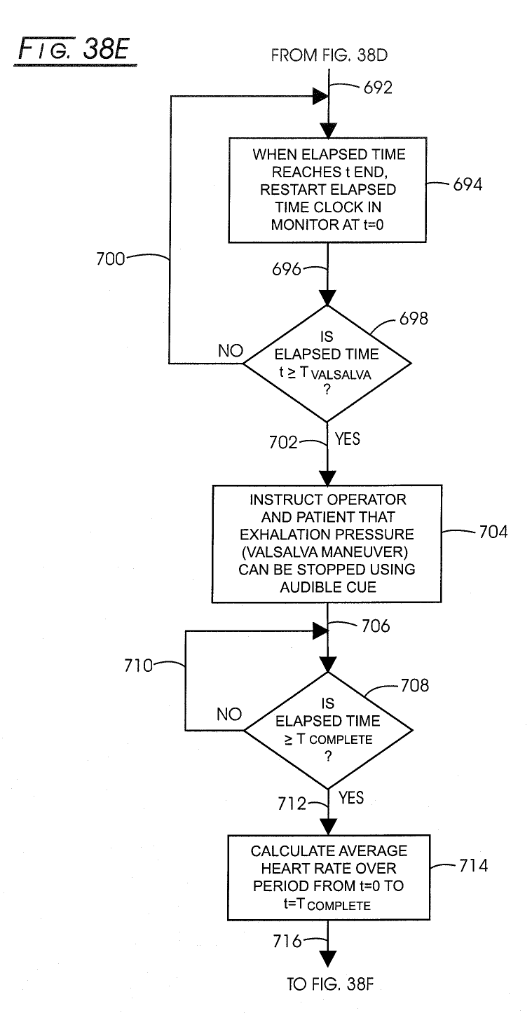

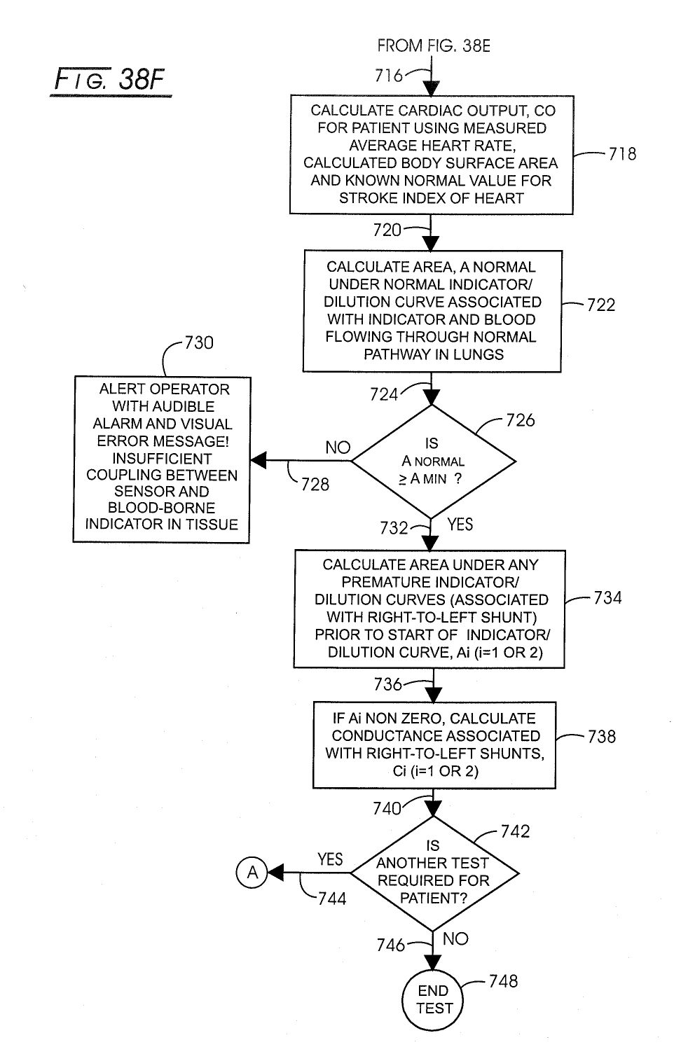

FIGS. 38A-38F combine with labels thereon to provide a flow chart illustrating the method and system at hand. Looking to FIG. 38A, the system starts at node 580 and continues as represented at arrow 582 to block 584. Block 584 is concerned with initialization, for example, setting the system default parameters and setting the PFLAG equal to zero. Next, as represented at arrow 586 and block 588, patient identification is recorded, patient position as either sitting or supine is determined, the height of the patient, the weight of the patient, the patient's sex and recommended injectate volume for circulatory indicator reagent is compiled. As represented at arrow 590 and block 592, from the information at block 588, body surface area, BSA, is calculated using the above-described Mosteller formula.

The indicator ICG typically is provided to the practitioner in solid or particulate form. Accordingly, it is necessary to mix it with sterile water as represented at arrow 594 and block 596, the indicator solution is prepared by mixing a known weight of indocyanine green dye with a predetermined volume of diluents such as sterile water or phosphate buffered saline. A predetermined volume of that indicator, for example, 2 ml drawn into a first syringe as described at 120 in FIG. 8. Additionally, as represented at arrow 598 and block 600 a second syringe as described at 122 in FIG. 8 is filled with a predetermined volume of isotonic saline to be used to “flush” the introducing tubing, venous catheter and peripheral vein so that all injected indicators are promptly delivered to the right atrium of the heart.

The thus filled two syringes are connected to two ports on the three-way valve as identified at 124 in FIG. 8. In this regard, arrow 602 extends to block 604 setting forth this valve connection. The procedure then continues as represented at arrow 606 which reappears in the FIG. 38B extending to block 608 where the relatively short vein access catheter 132 (FIG. 8) placed in a peripheral vein, i.e., antecubital vein in an arm. Additionally, flow sensor 126 is attached at the terminus of the extension tubing with three-way valve 124 pre-attached to the proximal end of the flow sensor. The three-way valve is turned in the direction of the flow sensor. Additionally, as represented at arrow 610 and block 612 an indicator sensor is positioned at the site of a blood vessel, for example, on the surface of skin at the pinna of the ear. That device is shown at 110 in FIG. 8. Practitioners generally will wish to apply an optical gel between the surface of the ear and the sensor to improve photonic transfer. Additionally, the fingerclip heart rate monitor is applied to the finger of the patient as shown at 114 in FIG. 8.

The invention is embodied also in a kit providing a clinician with the disposable materials utilized when practicing the new method. A kit supplying consumable materials necessary for quantifying a circulatory anomaly typically would include a dose of circulatory indicator reagent as a shelf stable material; a diluent for preparing the dose of circulatory indicator reagent for injection; a syringe and needle apparatus for mixing the dose of circulatory indicator reagent and the diluent and suitable for injecting the dose into an injection port; and a dose of nonreactive blood compatible clearing reagent for completing the injection.

As represented at arrow 614 and block 616, a determination is made as to whether the test at hand will be performed with a Valsalva Maneuver. In the event that such maneuver will not be used, then the method diverts as represented at arrow 618. Where the Valsalva Maneuver is to be used, then as represented at arrow 620 and block 622, the mouthpiece of the pressure transducer or manometer is set in the mouth of the patient as shown at 106 in FIG. 8 and is further connected to the receptacle at monitor 82. As represented at arrow 624 and symbol 626, the measurement start is at hand and the method continues as represented at arrow 628. Arrow 628 reappears in FIG. 38C extending to block 630 providing for the start of measurement of heart rate. Next, the patient as represented at arrow 632 and block 634, the patient is instructed to begin the Valsalva Maneuver by exhaling into the mouthpiece to reach and continue a minimum level of 40 mmHg of pressure. The patient will have been instructed that this breathing maneuver will be continuous for about 15-20 seconds. Meanwhile, the patient as well as the practitioner may observe the dynamic bar graph at the display of the monitoring tube. This continuous measurement is represented at arrow 636 and block 638. The system carries out this continuous monitoring looking for the noted minimum exhalation pressure as represented at arrow 640 and block 642. In the event that minimum pressure falls below the threshold, that event is then represented at arrow 644 and block 646. An audible alarm is sounded and a visual error message published instructing the patient to increase pressure. PFLAG is set to zero and the program reverts as represented at arrow 648 to arrow 632 where the system responds leading to the recommencement of the timing of the Valsalva Maneuver. Where the exhalation pressure is maintained at an appropriate level, then as represented at arrow 650 and block 652 PFLAG is set to one and the program continues as represented at arrow 654. Returning momentarily to FIG. 38B, and the query posed at block 616, where the test is not to be performed with a Valsalva Maneuver, the program reverted to arrow 618 which reappears in FIG. 38C extending to block 656 which sets PFLAG at two and reverts to arrow 654 as represented at arrow 658. Next, as represented at block 660 the elapsed time clock is started at time, t=0, and the program continues as represented at arrow 662.

Arrow 662 reappears in FIG. 38D extending to block 664 providing for the start of measurement of concentration of indicator. Next, as represented at arrow 666 and block 668, a query is posed as to whether, t, is at the time T_{inject}. In the event it is not, then the system dwells as represented at arrow 670 extending to arrow 636. However, where the time to inject is at hand, then as represented at arrow 672 and block 674, the operator is instructed with an audible cue to inject an indicator bolus from the first syringe and immediately inject isotonic saline flush bolus from the second syringe. The three-way valve will be manipulated during these injections. Also during the injections, the fluid flow sensor 126 will be active as represented at arrow 676 and block 678. The start and end of injection is measured with the flow sensor. A check is made as represented at arrow 680 and block 682 determining whether the start time less the end time is greater than the time of injection. Where it is not, then as represented at arrow 684 and block 686, a negative response causes the operator to be audibly alarmed with a visual error message that the injection was not completed in the allowed time interval. It is restarted as represented at arrow 688 leading to node A. Node A reappears in FIG. 38B along with arrow 690 extending to line 613 leading to a query as to whether the test is to be performed with a Valsalva Maneuver.

Where the determination at block 682 is that the injection took place in a proper time frame, the program continues as represented at arrow 692. Arrow 692 reappears in FIG. 38E extending to block 694. When the elapsed time is extended the elapsed time clock is restarted at t=0. As represented at arrow 696 query is posed as to whether the elapsed time is greater than or equal to the time for Valsalva Maneuver continuation. Where the answer to this query is in the negative, then the program dwells as represented at arrow 700 extending to arrow 692. Where the determination made with respect to the query posed at block 698 is in the affirmative, then as represented at arrow 702 and block 704, the operator and patient are audibly cued that the Valsalva Maneuver can be stopped. Next, as represented at arrow 706 and block 708, a determination is made as to whether the elapsed time is greater than or equal to the completion time. In the event it is not, then the program dwells as represented at arrow 710 extending to arrow 706. Where the elapsed time has reached the time of completion, then as represented at arrow 712 and block 714, average heart rate over the elapsed time is computed and the program continues as represented at arrow 716. Arrow 716 reappears in FIG. 38F extending to block 718. Block 718 calls for calculating cardiac output using the measured average heart rate, calculated body surface area and known normal value for stroke index of the heart. Next, as represented at arrow 720 and block 722, the area, A, under the normal indicator dilution curve is calculated (AUC). This is the curve representing blood and indicator flowing through the lungs. Next, as represented at arrow 724 and block 726, a query posed at block 726 determines whether the calculated normal area is greater than or equal to a minimum under curve area. In the event that it is not, then as represented at arrow 728 and block 730, the operator is alerted with an audible alarm and visual error message that insufficient coupling exists between the sensor and blood-borne indicator in tissue. Where a query at block 726 results in an affirmative determination, then the area under any premature indicator/dilution curve or curves associated with a right-to-left shunt is/are calculated. The parameter Ai is the area under premature indicator dilution/curve, i, where i is either one or two. Next, as represented at arrow 736 and block 738, if Ai is non-zero, then the conductance associated with right-to-left shunts is calculated as described herein. Then, as represented at arrow 740 and block 742, a query is made as to whether another test is required for this patient. In the event of an affirmative determination, then as represented at arrow 744, node A reappearing in FIG. 38B is reassessed. In the event of a negative determination to the query posed at block 742 then is represented at arrow 746 and symbol 748, the test is ended.

While the invention has been described with reference to preferred embodiments, those skilled in the art will understand that various changes may be made and equivalents may be substituted for elements thereof without departing from the scope of the invention. In addition, many modifications may be made to adapt a particular situation or material to the teachings of the invention without departing from the essential scope thereof. Since certain changes may be made in the above compositions and methods without departing from the scope of the invention herein involved, it is intended that all matter contained in the above descriptions and examples or shown in the accompanying drawings shall be interpreted as illustrative and not in a limiting sense. In this application all units are in the metric system or English system for the case of dimensions of the flow sensor 126 and its components and all amounts and percentages are by weight, unless otherwise expressly indicated. Also, all citations referred herein are expressly incorporated herein by reference. All terms not specifically defined herein are considered to be defined according to Dorland's Medical Dictionary, and if not defined therein according to Webster's New Twentieth Century Dictionary Unabridged, Second Edition. The disclosures of all of the citations provided are being expressly incorporated herein by reference. The disclosed invention advances the state of the art and its many advantages include those described and claimed.

What is claimed:

1. A method for detecting the presence of a right-to-left pulmonary shunt in a patient, the improvement comprising the steps:
providing an elongate air conduit having proximal end configured to be positioned within the mouth of the patient to receive exhaled breath, and having a distal end configured for air pressure secure attachment with a pressure transducer;
providing an indicator delivery system having an outlet located in a vein of the patient in blood flow communication with the right side of the heart of the patient and actuateable by an operator to inject a predetermined amount of metabolically compatible indicator into the vein for travel to the right side of the heart of the patient;
providing an indicator sensor located with respect to arterial vasculature and having an output corresponding with the instantaneous concentration of indicator at such vasculature; and
providing a monitor/controller having a display and supporting the pressure transducer in attachment with the air conduit distal end and having a pressure output signal, responsive to such pressure output signal reaching and at least maintaining a predetermined corresponding pressure threshold value to provide a visual representation thereof at the display and commence a test timing, responsive to the test timing to cue the operator to actuate the indicator delivery system, responsive to the sensor output to determine whether a shunt is present.

2. The method of claim 1 in which:
the elongate air conduit is provided as a flexible polymeric tube promoting a buffering of air pressure variations therein.

3. The method of claim 1 in which:
the monitor/controller is provided with a display with a visible dynamic component response to the pressure output signal to derive a visible cue relating such pressure output with the threshold value.

4. The method of claim 1 in which:
the monitor/controller is provided with a visible dynamic component present as a dynamic bar graph.

5. The method of claim 1 in which:
the monitor/controller is provided being responsive to a pressure output signal representing a drop in air pressure dropping below the pressure threshold value to create an aural warning.

6. The method of claim 1 in which:
the monitor/controller is provided to respond to the maintenance of the pressure output signal at least representing the pressure threshold value and to the commencement of test timing to provide an audible cue to the operator to actuate the indicator delivery system.

7. The method of claim 6 in which:
the indicator delivery system is provided to include a flow sensor responsive to identify the commencement and termination of fluid flow through the system; and
the monitor/controller is provided to respond to said commencement and termination of fluid flow and derive an audible alarm to the operator where the time interval of indicator injection is excessive.

8. The method of claim 1 in which:
the monitor/controller is provided as being responsive to the pressure output signal reaching and at least maintaining the corresponding pressure threshold value to commence timing a Valsalva Maneuver interval and is responsive to the termination of that interval to provide an audible cue of such termination.

9. The method of claim 1 further comprising the step:
providing a heart rate monitor having a heart rate output corresponding with the heart rate of the patient;
the monitor/controller is provided as being responsive to calculate cardiac output using an average of the heart rate output, body surface area of the patient and known normal value for stroke index of heart.

10. The method of claim 9 in which:
the monitor/controller is provided as being responsive to calculate the area Aₙₒᵣₘₐₗ under a normal indicator/dilution curve as associated with indicator and blood flowing through a normal pathway through and from the lungs of the patient.

11. The method of claim 10 in which:
the monitor/controller is provided as being responsive to compare the calculated area Aₙₒᵣₘₐₗ with a minimum value area, Aₘᵢₙ and is responsive to generate an audible alarm, error message and prompt when Aₙₒᵣₘₐₗ is less than Aₘᵢₙ.

12. The method of claim 10 in which:
the monitor/controller is provided as being responsive to calculate the area under any premature indicator/dilution curve associated with one or more right-to-left shunts which occur prior to the normal indicator/dilutive curve.

13. The method of claim 12 in which:
the monitor/controller is provided as being responsive to publish the normal indicator/dilution curve and any premature indicator/dilution curves at its display.

14. The method of claim 12 in which:
the monitor/controller is provided as being responsive to calculate the conductance associated with premature indicator/dilution curves.

15. A system for detecting the presence of one or more right-to-left cardiac shunts in a patient, comprising;
an indicator delivery assembly having an injection outlet located in a vein of the patient in blood flow communication with the right side of the heart of the patient and actuateable by an operator to inject a predetermined amount of indicator into the vein for travel to the right side of the heart of the patient;
an indicator sensor located with respect to arterial vasculature and having a concentrator output corresponding with the instantaneous concentration of indicator at such vasculature;
a heart rate monitor attachable in monitoring relationship with the patient, having a heart rate output correspond with the heart rate of the patient and a monitor/controller having a display, responsive to commence timing for test elapsed time and cue the operator to actuate the indicator delivery assembly, responsive to the concentrator output, the average heart rate output, the body surface area of the patient, and known normal value for the stroke index of the heart to calculate cardiac output, responsive to calculate the area Aₙₒᵣₘₐₗ under a normal indicator/dilution curve as associated with indicator and blood flowing through a normal pathway through and from the lungs of the patient, and responsive to calculate the area under any premature indicator/dilution curve associated with one or more right-to-left shunts which occur prior to the normal indicator/dilution curve.

16. The system of claim 15 in which:
the monitor/controller is responsive to publish the normal indicator/dilution curve and any premature indicator/dilution curves at its display.

17. The system of claim 15 in which:
the monitor/controller is responsive to fluid calculate the conductive associated with premature indicator/dilution curve.

18. The system of claim 15 in which:
the monitor/controller is responsive to compare the calculated area A normal with a minimum value area Aₘᵢₙ, and is responsive to generate an audible alarm, error message and prompt when A normal is less than Aₘᵢₙ.

19. The system of claim 15 in which:
the indicator delivery system includes a flow sensor responsive to derive signals corresponding with the commencement and termination of fluid flow through the system; and
the monitor/controller is responsive to such commencement and termination signals to derive an audible alarm to the operator when the time interval of indicator injection is excessive.

20. The system of claim 15 in which:
the indicator delivery assembly is actuateable to inject a fluorescing biocompatible dye excitable by tissue penetrating excitation radiation to derive fluorescence emission corresponding with the indicator concentration; and
the sensor comprises a photodiode energizable to generate light at the excitation radiation wavelength and a photodetector which is filtered for response substantially only to the fluorescence emission.

21. The system of claim 20 in which:
the sensor is configured for transcutaneous performance of the photodiode and the filtered photodetectors.

22. The system of claim 20 in which:
the sensor comprises two or more paired filtered photodetector and excitation photodiodes energizable in a sequence of such pairs; and
the monitor/controller is responsive to elect that pair exhibiting a concentrator output of highest intensity.

23. The system of claim 20 in which:
the indicator delivery assembly injected indicator is indocyanine green dye.

24. The system of claim 23 in which:
the sensor excitation photodiode is energizable to emit light at a wavelength of 785 nanometers.

25. The system of claim 15 in which:
the indicator delivery assembly includes a flow sensor configured as fluid transmitting tube surmounted in heat transfer relationship by an electrical resistance heater having a heat responsive resistance control deriving signals corresponding with the commencement and termination of fluid flow through the system.

26. The system of claim 1 in which:
the indicator delivery assembly comprises a flexible elongate delivery tube extending between proximal and distal ends, an auxiliary catheter coupled in fluid transfer relationship with the distal end defining the outlet, a fluid flow sensor coupled in fluid transfer relationship with the proximal end and deriving signals corresponding with the commencement and termination of fluid flow through the system, a valve connected upstream to the fluid flow sensor, a first indicator containing syringe coupled in indicator flow relationship with the valve and actuateable to cause indicator to flow through the valve, and a second isotonic saline fluid containing syringe coupled in fluid flow relationship with the valve and actuateable to cause isotonic saline to flow through the valve; and
the monitor/controller is responsive to cue the operator first to actuate the first syringe and immediately thereafter to actuate the second syringe, and is responsive to monitor the corresponding fluid flow sensor signals.

27. A method for quantifying the extent of a circulatory anomaly in a patient comprising the steps;
an indicator delivery assembly having an injection outlet located in a vein of the patient in blood flow communication with the right side of the heart of the patient and actuateable by an operator to inject a predetermined indicator bolus into the vein for travel to the right side of the heart of the patient;
an indicator sensor located with respect to arterial vasculature and having a concentrator output corresponding with the instantaneous concentration of indicator at such vasculature;
a heart rate monitor attachable in monitoring relationship with the patient, having a heart rate output correspond with the heart rate of the patient and a monitor/controller having a display, responsive to commence timing for test elapsed time and cue the operator to actuate the indicator delivery assembly, responsive to the concentrator output, the average heart rate output, the body surface area of the patient, and known normal value for the stroke index of the heart to calculate cardiac output, responsive to calculate the area A normal under a normal indicator/dilution curve as associated with indicator and blood flowing through a normal pathway from the lungs of the patient, and responsive to calculate the area under any premature indicator/dilution curve associated with one or more right-to-left shunts which occur prior to the normal indicator/dilution curve.

28. The method of claim 27 further providing an elongate air conduit having proximal end configured to be positioned within the mouth of the patient to receive exhaled breath, and having a distal end configured for air pressure secure attachment with a pressure transducer;

29. The method of claim 28 wherein the method is performed with the imposition of a breathing maneuver to create a sufficient positive pressure differential between the right side and the left side of the heart to induce blood flow across a right-to-left shunt for a period of time immediately preceding the injection of the indicator bolus.

30. The method of claim 29 wherein a cue is provided for continuing a breathing maneuver after injection of the indicator bolus for about five seconds or the time necessary for the indicator bolus to travel through the heart.

31. The method of claim 27 in which:
the monitor/controller is provided as being responsive to calculate the area A normal under a normal indicator/dilution curve as associated with indicator and blood flowing through a normal pathway from the lungs of the patient;
the monitor/controller is provided as being responsive to compare the calculated area A normal with a minimum value area, Aₘᵢₙ and is responsive to generate an audible alarm, error message and prompt when A normal is less than Aₘᵢₙ; and
the monitor/controller is provided as being responsive to calculate the area under any premature indicator/dilution curve associated with one or more right-to-left shunts which occur prior to the normal indicator/dilutive curve.

32. The method of claim 27 wherein the indicator sensor is located at one or more of the skin, ear, lip, tongue, supraorbital artery on forehead, nose, face and carotid artery.

33. The method of claim 27 wherein the indicator bolus further comprises a predetermined amount of metabolically compatible indicator that is one or more of indocyanine green dye, Evans Blue dye, fluorescein, a fluorescing agent, ammonium chloride, lithium chloride, glucose, gas bubbles, echogenic particles, microspheres, radiographic contrast agents, angiography contrast agents, radioactive isotope, protein and heparin.

34. The method of claim 33 wherein the indicator is indocyanine green dye, detected transcutaneously through fluorescence measurement methods, and the amount of injected dye is within the range of about 0.5 milligram to 10 milligrams.

35. The method of claim 34 wherein the amount of injected dye is in the range of one or more of 1 to 5 milligrams, 0.5 to 7.5 milligram/milliliter, and 1 to 2.5 milligram/milliliter.

36. The method of claim 35 wherein the indicator is measured by one or more of spectroscopy, densitometry, radiometry, ultrasound, magnetic resonance methods and radiography methods.

37. The method of claim 27 wherein the circulatory anomaly is one or more of a patent foramen ovale, an intracardiac right-to-left shunt, a pulmonary arteriovenous malformation and an arteriovenous malformation.

38. A sensor apparatus comprising
(a) an emitter-detector pair for monitoring the fluorescence of a fluorescing circulatory tracking reagent;
b) said emitter providing a light source emitting a first wavelength for the transcutaneous excitation of an indicator within the bloodstream; and
(c) said detector for measuring the intensity of the light emitted at a second wavelength from an indicator within the blood stream.

39. The apparatus of claim 38 wherein a plurality of emitter and detector pairs are disposed in a sensor array.

40. The apparatus of claim 39 wherein the sensors are sequentially queried to determine the emitter and detector pair providing a preferred sensor from the sensor array.

41. The apparatus of claim 40 wherein the sensor is a transcutaneous sensor and the preferred sensor is determined by identifying the sensor in closest relationship a subcutaneous blood vessel in order to maximize the sensitivity of said sensor, wherein the preferred sensor is determined by one or more of signal to noise ratio, absolute signal level, and minimum background signal.

42. The apparatus of claim 41 wherein an optical coupling agent is utilized at the skin/instrument interface.

43. A kit supplying consumable materials necessary for quantifying a circulatory anomaly comprising a dose of circulatory indicator reagent as a shelf stable material; a diluent for preparing the dose of circulatory indicator reagent for injection; a syringe and needle apparatus for mixing the dose of circulatory indicator reagent and the diluent and suitable for injecting the dose into an injection port; and a dose of nonreactive blood compatible clearing reagent for completing the injection.

44. A method of determining the efficacy of circulatory tracking systems comprising
a) providing a test animal with a functioning circulatory system and heart;
b) emplacing an injection catheter into the circulatory system or a chamber of the heart in the test animal;
c) providing a test circulatory tracking reagent;
d) providing a detector compatible with the circulatory tracking reagent; and
e) one or more detector emplacement locations on the body of the test animal,
wherein a bolus of circulatory tracking reagent is injected into the circulatory system or chamber of the heart, and the detector is emplaced on the body of the test animal such that the efficacy of a combination of given detectors to detect the presence of given circulatory tracking agents at a particular location on the test animal body may be determined.
